(19) United States
(12) Patent Application Publication
(10) Pub. No.: US 2004/0005316 A1
(43) Pub. Date: Jan. 8, 2004

(54) Use of HMG fragments as anti-inflammatory agents

(75) Inventors: Kevin J. Tracey, Old Greenwich, CT (US); Huan Yang, Douglaston, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: North Shore-Long Island Jewish Research Institute, Manhasset, NY

(21) Appl. No.: 10/456,949

(22) Filed: Jun. 6, 2003

Related U.S. Application Data
(63) Continuation of application No. 10/147,447, filed on May 15, 2002.
(60) Provisional application No. 60/291,034, filed on May 15, 2001.

(51) Int. Cl. A61K 39/395

(52) U.S. Cl. 424/143.1

(57) ABSTRACT

Compositions and methods are disclosed for inhibiting the release of a proinflammatory cytokine from a vertebrate cell, and for inhibiting an inflammatory cytokine cascade in a patient. The compositions comprise a vertebrate HMG A box, and an antibody preparation that specifically binds to a vertebrate HMG B box. The methods comprise treating a cell or a patient with sufficient amounts of the composition to inhibit the release of the proinflammatory cytokine, or to inhibit the inflammatory cytokine cascade.

### RELATED APPLICATIONS

This application is a continuation of U.S. application Ser. No. 10/147,447, filed May 15, 2002, which claims the benefit of U.S. Provisional Application No. 60/291,034, filed May 15, 2001, the entire teachings which are incorporated herein by reference.

### GOVERNMENT SUPPORT

The invention was supported, in whole or in part, by a grant RO1 GM 57226-02 from the National Institutes of Health. The Government has certain rights in the invention.

### BACKGROUND OF THE INVENTION

Inflammation is often induced by proinflammatory cytokines, such as tumor necrosis factor (TNF), interleukin (IL)-1α, IL-1β, IL-6, platelet-activating factor (PAF), macrophage migration inhibitory factor (MIF), and other compounds. These proinflammatory cytokines are produced by several different cell types, most importantly immune cells (for example, monocytes, macrophages and neutrophils), but also non-immune cells such as fibroblasts, osteoblasts, smooth muscle cells, epithelial cells, and neurons. These proinflammatory cytokines contribute to various disorders during the early stages of an inflammatory cytokine cascade.

Inflammatory cytokine cascades contribute to deleterious characteristics, including inflammation and apoptosis, of numerous disorders. Included are disorders characterized by both localized and systemic reactions, including, without limitation, diseases involving the gastrointestinal tract and associated tissues (such as appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, coeliac disease, hepatitis, Crohn's disease, enteritis, and Whipple's disease); systemic or local inflammatory diseases and conditions (such as asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, and sarcoidosis); diseases involving the urogenital system and associated tissues (such as septic abortion, epididymitis, vaginitis, prostatitis, and urethritis); diseases involving the respiratory system and associated tissues (such as bronchitis, emphysema, rhinitis, cystic fibrosis, pneumonitis, adult respiratory distress syndrome, pneumoultramicroscopicsilicovolcanoconiosis, alvealitis, bronchiolitis, pharyngitis, pleurisy, and sinusitis); diseases arising from infection by various viruses (such as influenza, respiratory syncytial virus, HIV, hepatitis B virus, hepatitis C virus and herpes), bacteria (such as disseminated bacteremia, Dengue fever), fungi (such as candidiasis) and protozoal and multicellular parasites (such as malaria, filariasis, amebiasis, and hydatid cysts); dermatological diseases and conditions of the skin (such as burns, dermatitis, dermatomyositis, sunburn, urticaria warts, and wheals); diseases involving the cardiovascular system and associated tissues (such as vasulitis, angiitis, endocarditis, arteritis, atherosclerosis, thrombophlebitis, pericarditis, congestive heart failure, myocarditis, myocardial ischemia, periarteritis nodosa, and rheumatic fever); diseases involving the central or peripheral nervous system and associated tissues (such as Alzheimer's disease, meningitis, encephalitis, multiple sclerosis, cerebral infarction, cerebral embolism, Guillame-Barre syndrome, neuritis, neuralgia, spinal cord injury, paralysis, and uveitis); diseases of the bones, joints, muscles and connective tissues (such as the various arthritides and arthralgias, osteomyelitis, fasciitis, Paget's disease, gout, periodontal disease, rheumatoid arthritis, and synovitis); other autoimmune and inflammatory disorders (such as myasthenia gravis, thryoiditis, systemic lupus erythematosus, Goodpasture's syndrome, Behcets's syndrome, allograft rejection, graft-versus-host disease, Type I diabetes, ankylosing spondylitis, Berger's disease, and Retier's syndrome); as well as various cancers, tumors and proliferative disorders (such as Hodgkins disease); and, in any case the inflammatory or immune host response to any primary disease.

The early proinflammatory cytokines (e.g., TNF, IL-1, etc.) mediate inflammation, and induce the late release of high mobility group-1 (HMG1) (also known as HMG-1 and HMGB1), a protein that accumulates in serum and mediates delayed lethality and further induction of early proinflammatory cytokines.

HMG1 was first identified as the founding member of a family of DNA-binding proteins termed high mobility group (HMG) that are critical for DNA structure and stability. It was identified nearly 40 years ago as a ubiquitously expressed nuclear protein that binds double-stranded DNA without sequence specificity.

HMG1 binding bends DNA to promote formation and stability of nucleoprotein complexes that facilitate gene transcription of glucocorticoid receptors and RAG recombinase. The HMG1 molecule has three domains: two DNA binding motifs termed HMG A and HMG B boxes, and an acidic carboxyl terminus. The two HMG boxes are highly conserved 80 amino acid, L-shaped domains. HMG boxes are also expressed in other transcription factors including the RNA polymerase I transcription factor human upstream-binding factor and lymphoid-specific factor.

Recent evidence has implicated HMG1 as a cytokine mediator of delayed lethality in endotoxemia. That work demonstrated that bacterial endotoxin (lipopolysaccharide (LPS)) activates monocytes/macrophages to release HMG1 as a late response to activation, resulting in elevated serum HMG1 levels that are toxic. Antibodies against HMG1 prevent lethality of endotoxin even when antibody administration is delayed until after the early cytokine response. Like other proinflammatory cytokines, HMG1 is a potent activator of monocytes. Intratracheal application of HMG1 causes acute lung injury, and anti-HMG1 antibodies protect against endotoxin-induced lung edema. Serum HMG1 levels are elevated in critically ill patients with sepsis or hemorrhagic shock, and levels are significantly higher in non-survivors as compared to survivors.

HMG1 has also been implicated as a ligand for RAGE, a multi-ligand receptor of the immunoglobulin superfamily. RAGE is expressed on endothelial cells, smooth muscle cells, monocytes, and nerves, and ligand interaction transduces signals through MAP kinase, P21 ras, and NF-KB. The delayed kinetics of HMG1 appearance during endotoxemia makes it a potentially good therapeutic target, but little is known about the molecular basis of HMG1 signaling and toxicity.

Therefore, it would be useful to identify characteristics of HMG1 proinflammatory activity, particularly the active domain(s) responsible for this activity, and any inhibitory effects of other domains.

### SUMMARY OF THE INVENTION

The present invention is based on the discoveries that (1) the HMG A box serves as a competitive inhibitor of HMG proinflammatory action, and (2) the HMG B box has the predominant proinflammatory activity of HMG.

Accordingly, the present invention is directed to a polypeptide comprising a vertebrate HMG A box or a biologically active fragment thereof or a non-naturally occurring HMG A box or a biologically active fragment thereof. The HMG A box or these embodiments can inhibit release of a proinflammatory cytokine from a vertebrate cell treated with HMG. The HMG A box is preferably a mammalian HMG A box, more preferably, a mammalian HMG1 A box, for example, a human HMG1 A box, and most preferably, the HMG1 A box comprising or consisting of the sequence of SEQ ID NO:4 or SEQ ID NO:22. In a preferred embodiment, the vertebrate cell is a mammalian macrophage. The present invention also encompasses vectors encoding these polypeptides.

In other embodiments, the invention is directed to a composition comprising the HMG A box polypeptide or a biologically active fragment thereof described above in a pharmaceutically acceptable excipient. In these embodiments, the composition can inhibit a condition characterized by activation of an inflammatory cytokine cascade. The composition can further comprise an antagonist of an early sepsis mediator. The antagonist of an early sepsis mediator is preferably an antagonist of a cytokine selected from the group consisting of TNF, IL-1α, IL-1β, MIF and IL-6, more preferably, an antibody to TNF or MIF, or an IL-1 receptor antagonist.

In these embodiments, the condition is preferably selected from the group consisting of appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopicsilicovolcanoconiosis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, Type I diabetes, ankylosing spondylitis, Berger's disease, Retier's syndrome, and Hodgkins disease. More preferably, the condition is selected from the group consisting of appendicitis, peptic, gastric and duodenal ulcers, peritonitis, pancreatitis, ulcerative, pseudomembranous, acute and ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, coeliac disease, congestive heart failure, adult respiratory distress syndrome, cerebral infarction, cerebral embolism, spinal cord injury, paralysis, allograft rejection and graft-versus-host disease; most preferably, the condition is endotoxic shock or allograft rejection. When the condition is allograft rejection, the composition can further comprise an immunosuppressant used to inhibit allograft rejection, preferably cyclosporin.

In additional embodiments, the invention is directed to a purified preparation of antibodies that specifically bind to a vertebrate high mobility group protein (HMG) B box but do not specifically bind to non-B box epitopes of HMG. In these embodiments, the antibodies can inhibit a biological activity of an HMG B box polypeptide, for example, the release of a proinflammatory cytokine from a vertebrate cell treated with HMG. In preferred embodiments, the HMG B box is a mammalian HMG B box, for example, a human HMG B box, more preferably an HMG1 B box, most preferably the HMG1 B box with the amino acid sequence of SEQ ID NO:5 or SEQ ID NO:20. In another embodiment, the antibodies bind a specific polypeptide sequence of the HMG1 B box, comprising amino acids 1-20 of SEQ ID NO:20 (SEQ ID NO:16), or comprising amino acids 1-20 of SEQ ID NO:5 (SEQ ID NO:23), or consisting of amino acids 1-20 of SEQ ID NO:20 (SEQ ID NO:16), or consisting of amino acids 1-20 of SEQ ID NO:5 (SEQ ID NO:23). The vertebrate cell is also preferably a mammalian macrophage. In some embodiments, the antibodies are preferably humanized.

In additional embodiments, the invention is directed to a composition comprising any of the antibody preparations described above, in a pharmaceutically acceptable excipient. In these embodiments, the composition can inhibit a condition characterized by activation of an inflammatory cytokine cascade. These compositions can also usefully comprise an antagonist of an early sepsis mediator, as previously described. The preferred conditions useful for treatment with these compositions are those mediated or characterized by activation of an inflammatory cytokine cascade, for example, those conditions as enumerated with the A box compositions previously described.

Additionally, the present invention is directed to a polypeptide comprising a vertebrate HMG B box or a biologically active fragment thereof or a non-naturally occurring HMG B box or biologically active fragment thereof, but not comprising a full length HMG protein. In these embodiments, the polypeptide can cause release of a proinflammatory cytokine from a vertebrate cell. The polypeptide of these embodiments is preferably an HMG B box, more preferably an HMG1 B box, most preferably the HMG1 B box with the amino acid sequence given as SEQ ID NO:5 or SEQ ID NO:20. In another embodiment, the HMG B box fragment comprises the sequence of SEQ ID NO:16 or SEQ ID NO:23 or consists of the sequence of SEQ ID NO:16 or SEQ ID NO:23. In a preferred embodiment, the vertebrate cell is a mammalian macrophage. The present invention also encompasses vectors encoding these polypeptides.

The present invention is also directed to a method of inhibiting release of a proinflammatory cytokine from a mammalian cell. The method comprises treating the cell with either the A box or A box biologically active fragment polypeptide composition described above or the B box or B box biologically active fragment antibody compositions described above, in an amount sufficient to inhibit release of the proinflammatory cytokine from the cell. In these embodiments, the cell is preferably a macrophage. In addition, the proinflammatory cytokine is preferably selected from the group consisting of TNF, IL-1α, IL-1β, MIF and IL-6. More preferably the cell is a macrophage and the proinflammatory cytokine is preferably selected from the group consisting of TNF, IL-1α, IL-1β, MIF and IL-6. The methods preferably treat a cell in a patient suffering from, or at risk for, a condition characterized by activation of the inflammatory cytokine cascade. Preferred conditions have been enumerated previously.

In related embodiments, the present invention is directed to a method of treating a condition in a patient characterized by activation of an inflammatory cytokine cascade. The method comprises administering to the patient any of the A box or A box biologically active fragment polypeptide compositions or the B box or B box biologically active fragment antibody compositions described above in an amount sufficient to inhibit the inflammatory cytokine cascade. Preferred conditions have already been enumerated.

Additional embodiments are directed to a method of stimulating the release of a proinflammatory cytokine from a cell. The method comprises treating the cell with the B box polypeptide or a biologically active fragment thereof, or the vector of the B box polypeptide or B box biologically active fragment previously described in an amount sufficient to stimulate the release of the proinflammatory cytokine. In related embodiments, the invention is directed to a method for effecting weight loss or treating obesity in a patient. The method comprises administering to the patient an effective amount of the HMG B box polypeptide or a biologically active fragment thereof to the patient. In one embodiment, the HMG B box polypeptide or a biologically active fragment thereof is in a pharmaceutically acceptable excipient.

The present invention is also directed to a method of determining whether a compound inhibits inflammation. The method comprises combining the compound with (a) a cell that releases a proinflammatory cytokine when exposed to a vertebrate HMG B box or biologically active fragment thereof; and (b) the HMG B box or biologically active fragment thereof, then determining whether the compound inhibits the release of the proinflammatory cytokine from the cell. Preferably, the HMG B box is a mammalian HMG B box, for example, an HMG1 B box. Preferred proinflammatory cytokines are as previously described.

### BRIEF DESCRIPTION OF THE DRAWINGS

FIG. 1 is a schematic representation of HMG1 mutants and their activity in TNF release (pg/ml).

FIG. 2A is a histogram showing the effect of 0 μg/ml, 0.01 μg/ml, 0.1 μg/ml, 1 μg/ml or 10 μg/ml of B box on TNF release (pg/ml) in RAW 264.7 cells.

FIG. 2B is a histogram showing the effect of 0 μg/ml, 0.01 μg/ml, 0.1 μg/ml, 1 μg/ml or 10 μg/ml of B box on IL-1β release (pg/ml) in RAW 264.7 cells.

FIG. 2C is a histogram showing the effect of 0 μg/ml, 0.01 μg/ml, 0.1 μg/ml, 1 μg/ml or 10 μg/ml of B box on IL-6 release (pg/ml) in RAW 264.7 cells.

FIG. 2D a scanned image of a blot of an RNAse protection assay, showing the effect of B box (at 0 hours, 4 hours, 8 hours, or 24 hours after administration) or vector alone (at 4 hours after administration) on TNF mRNA expression in RAW 264.7 cells.

FIG. 2E is a histogram of the effect of HMG1 B box on TNF protein release (pg/ml) from RAW 264.7 cells at 0 hours, 4 hours, 8 hours, 24 hours, 32 hours or 48 hours after administration.

FIG. 2F is a histogram of the effect of vector on TNF protein release (pg/ml) from RAW 264.7 cells at 0 hours, 4 hours, 8 hours, 24 hours, 32 hours or 48 hours after administration.

FIG. 3 is a schematic representation of HMG1 B box mutants and their activity in TNF release (pg/ml).

FIG. 4A is a graph of the effect of 0 μg/ml, 5 μg/ml, 10 μg/ml, or 25 μg/ml of HMG1 A box protein on the release of TNF (as a percent of HMG1 mediated TNF release alone) from RAW 264.7 cells.

FIG. 4B is a histogram of the effect of HMG1 (0 or 1.5 μg/ml), HMG1 A box (0 or 10 μg/ml), or vector (0 or 10 μg/ml), alone, or in combination on the release of TNF (as a percent of HMG1 mediated TNF release alone) from RAW 264.7 cells.

FIG. 5A is a graph of binding of ¹²⁵I-HMGB1 binding to RAW 264.7 cells (CPM/well) over time (minutes).

FIG. 5B is a histogram of the binding of ¹²⁵I-HMGB1 in the absence of unlabeled HMGB1 or HMG1 A box for 2 hours at 4° C. (Total), or in the presence of 5,000 molar excess of unlabeled HMGB1 (HMGB1) or A box (A box), measured as a percent of the total CPM/well.

FIG. 6 is a histogram of the effects of HMG-1 (0 μg/ml or 1 μg/ml) or HMG1 B box (0 μg/ml or 10 μg/ml), alone or in combination with anti-B box antibody (25 μg/ml or 100 μg/ml) or IgG (25 μg/ml or 100 μg/ml) on TNF release from RAW 264.7 cells (expressed as a percent of HMG1 mediated TNF release alone).

FIG. 7A is a scanned image of a hematoxylin and eosin stained kidney section obtained from an untreated mouse.

FIG. 7B is a scanned image of a hematoxylin and eosin stained kidney section obtained from a mouse administered HMG1 B box.

FIG. 7C is a scanned image of a hematoxylin and eosin stained myocardium section obtained from an untreated mouse.

FIG. 7D is a scanned image of a hematoxylin and eosin stained myocardium section obtained from a mouse administered HMG1 B box.

FIG. 7E is a scanned image of a hematoxylin and eosin stained lung section obtained from an untreated mouse.

FIG. 7F is a scanned image of a hematoxylin and eosin stained lung section obtained from a mouse administered HMG1 B box.

FIG. 7G is a scanned image of a hematoxylin and eosin stained liver section obtained from an untreated mouse.

FIG. 7H is a scanned image of a hematoxylin and eosin stained liver section obtained from a mouse administered HMG1 B box.

FIG. 71 is a scanned image of a hematoxylin and eosin stained liver section (high magnification) obtained from an untreated mouse.

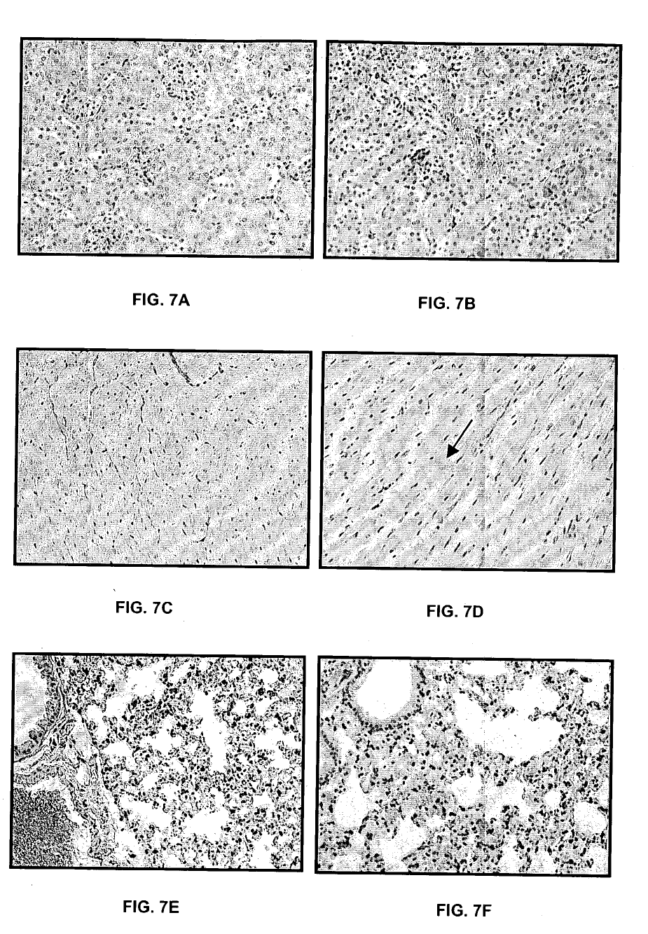

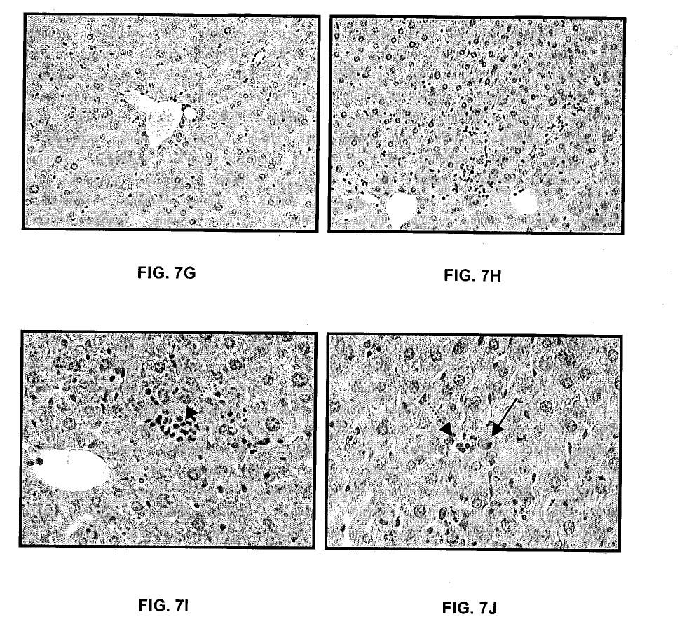

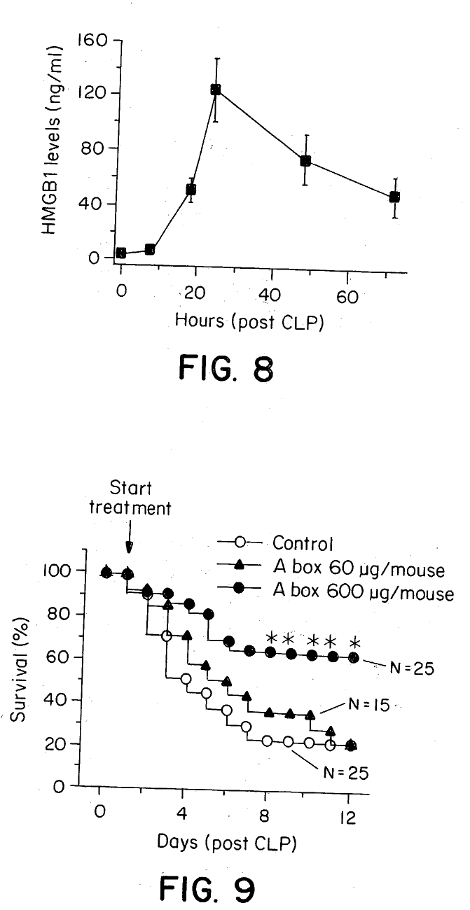

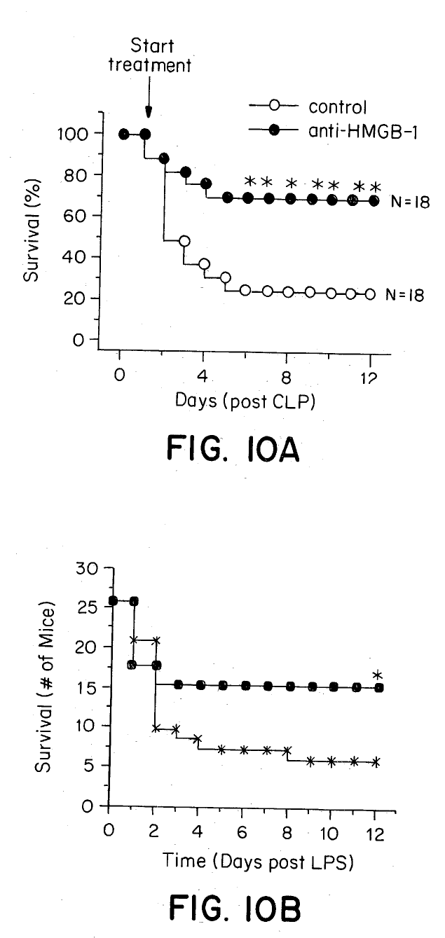

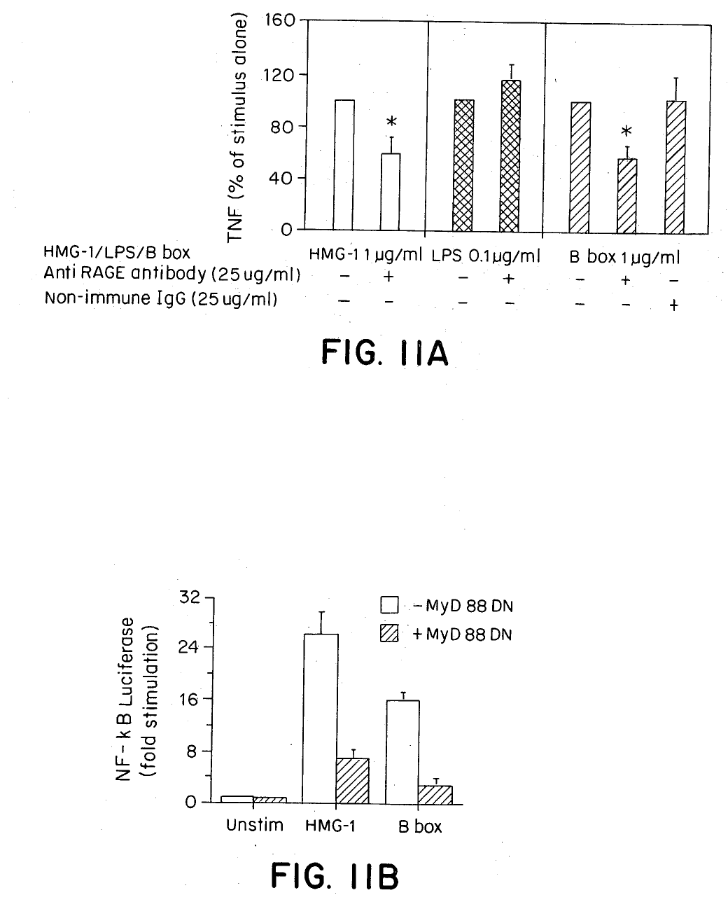

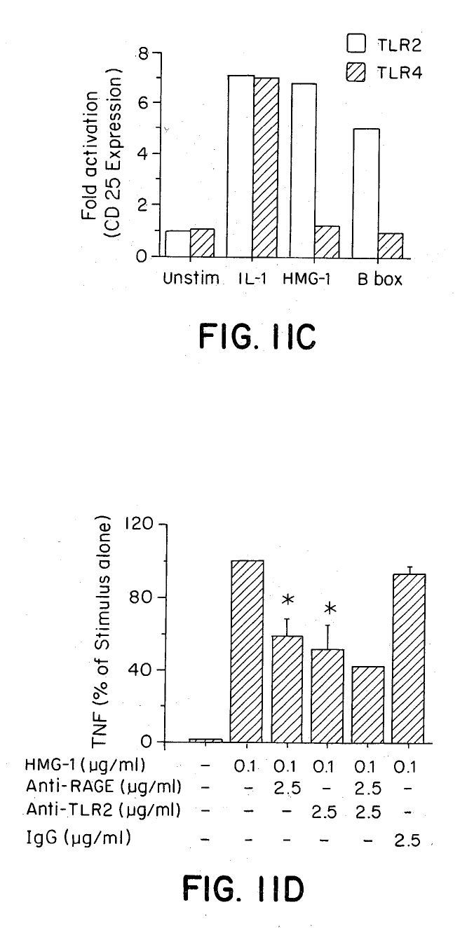

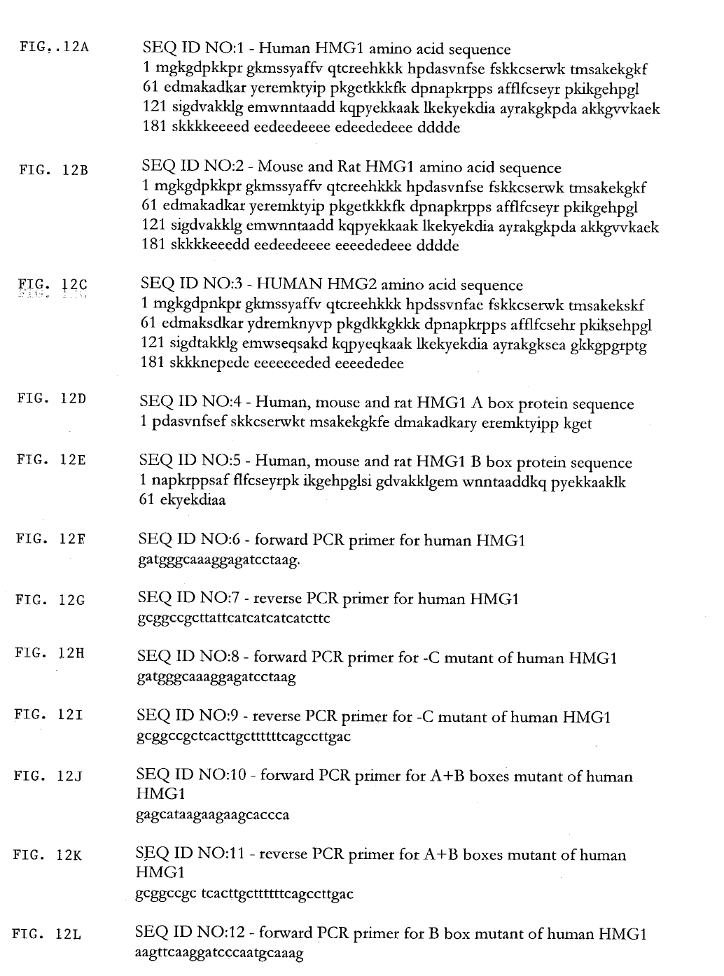

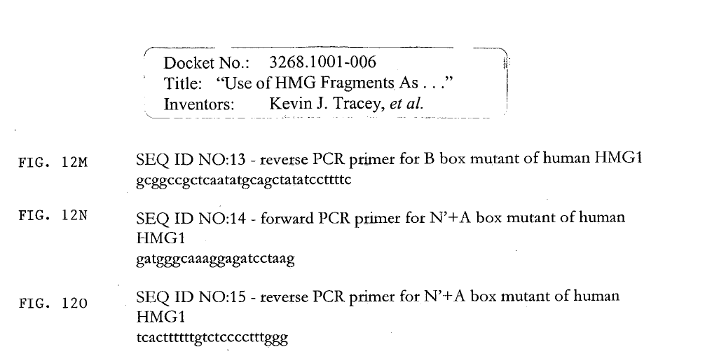

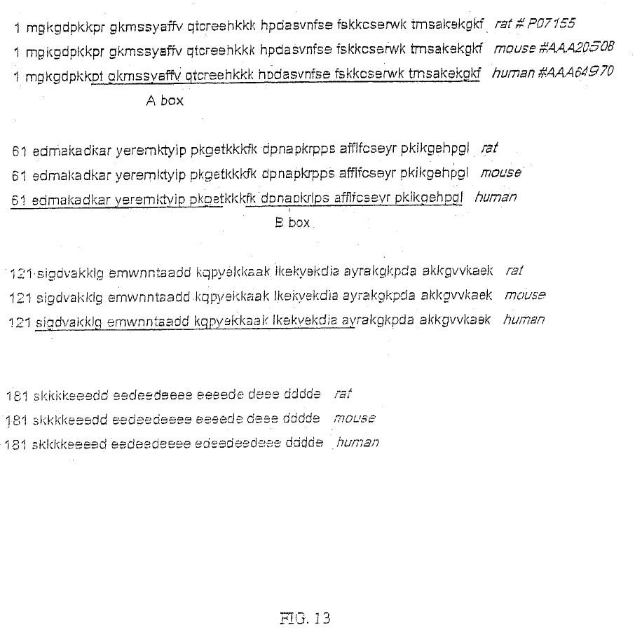

FIG. 7J is a scanned image of a hematoxylin and eosin stained liver section (high magnification) obtained from a mouse administered HMG1 B box.

FIG. 8 is a graph of the level of HMGB 1 (ng/ml) in mice subjected to cecal ligation and puncture (CLP) over time (hours).

FIG. 9 is a graph of the effect of A Box (60 μg/mouse or 600 μg/mouse) or no treatment on survival of mice over time (days) after cecal ligation and puncture (CLP).

FIG. 10A is a graph of the effect of anti-HMG1 antibody (dark circles) or no treatment (open circles) on survival of mice over time (days) after cecal ligation and puncture (CLP).

FIG. 10B is a graph of the effect of anti-HMG1 B box antiserum (▪) or no treatment (*) on the survival (days) of mice administered lipopolysaccharide (LPS).

FIG. 11A is a histogram of the effect of anti-RAGE antibody or non-immune IgG on TNF release from RAW 264.7 cells treated with HMG1 (HMG-1), lipopolysaccharide (LPS), or HMG1 B box (B box).

FIG. 11B is a histogram of the effect of HMG1 or HMG1 B box polypeptide stimulation on activation of the NF-kB-dependent ELAM promoter (measured by luciferase activity) in RAW 264.7 cells co-transfected with a murine MyD 88-dominant negative (+MyD 88 DN) mutant (corresponding to amino acids 146-296), or empty vector (−MyD 88 DN). Data are expressed as the ratio (fold-activation) of average luciferase values from unstimulated and stimulated cells (subtracted for background)+SD.

FIG. 11C is a histogram of the effect stimulation of CHO reporter cell lines that constitutively express human TLR2 (open bars) or TLR4 (shaded bars) with IL-1, HMG1, or HMG1 B box on CD25 expression. Data are expressed as the ratio (fold-activation) of the percent of CD25⁺ cells in unstimulated and stimulated cell populations that were gated to exclude the lowest 5% of cells based on mean FL1 fluorescence.

FIG. 11D is a histogram of the effect of administration of anti-RAGE antibody, anti-TLR2 antibody, anti-RAGE antibody and anti-TLR2 antibody together, or IgG on HMG1-mediated TNF release (measured as a percent of TNF release in the absence of antibody) in RAW 264.7 cells.

FIG. 12A is the amino acid sequence of a human HMG1 polypeptide (SEQ ID NO:1).

FIG. 12B is the amino acid sequence of rat and mouse HMG1 (SEQ ID NO:2).

FIG. 12C is the amino acid sequence of human HMG2 (SEQ ID NO:3).

FIG. 12D is the amino acid sequence of a human, mouse, and rat HMG1 A box polypeptide (SEQ ID NO:4).

FIG. 12E is the amino acid sequence of a human, mouse, and rat HMG1 B box polypeptide (SEQ ID NO:5).

FIG. 12F is the nucleic acid sequence of a forward primer for human HMG1 (SEQ ID NO:6).

FIG. 12G is the nucleic acid sequence of a reverse primer for human HMG1 (SEQ ID NO:7).

FIG. 12H is the nucleic acid sequence of a forward primer for the carboxy terminus mutant of human HMG1 (SEQ ID NO:8).

FIG. 12I is the nucleic acid sequence of a reverse primer for the carboxy terminus mutant of human HMG1 (SEQ ID NO:9).

FIG. 12J is the nucleic acid sequence of a forward primer for the amino terminus plus B box mutant of human HMG1 (SEQ ID NO:10).

FIG. 12K is the nucleic acid sequence of a reverse primer for the amino terminus plus B box mutant of human HMG1 (SEQ ID NO:11).

FIG. 12L is the nucleic acid sequence of a forward primer for a B box mutant of human HMG1 (SEQ ID NO:12).

FIG. 12M is the nucleic acid sequence of a reverse primer for a B box mutant of human HMG1 (SEQ ID NO:13).

FIG. 12N is the nucleic acid sequence of a forward primer for the amino terminus plus A box mutant of human HMG1 (SEQ ID NO:14).

FIG. 12O is the nucleic acid sequence of a reverse primer for the amino terminus plus A box mutant of human HMG1 (SEQ ID NO:15).

FIG. 13 is a sequence alignment of HMG1 polypeptide sequence from rat (SEQ ID NO:2), mouse (SEQ ID NO:2), and human (SEQ ID NO:18).

### DETAILED DESCRIPTION OF THE INVENTION

The practice of the present invention will employ, unless otherwise indicated, conventional techniques of cell culture, molecular biology, microbiology, cell biology, and immunology, which are well within the skill of the art. Such techniques are fully explained in the literature. See, e.g., Sambrook et al., 1989, “Molecular Cloning: A Laboratory Manual”, Cold Spring Harbor Laboratory Press; Ausubel et al. (1995), “Short Protocols in Molecular Biology”, John Wiley and Sons; Methods in Enzymology (several volumes); Methods in Cell Biology (several volumes), and Methods in Molecular Biology (several volumes).

The present invention is based on a series of discoveries that further elucidate various characteristics of the ability of HMG1 to induce production of proinflammatory cytokines and inflammatory cytokine cascades. Specifically, it has been discovered that the proinflammatory active domain of HMG1 is the B box (and in particular, the first 20 amino acids of the B box), and that antibodies specific to the B box will inhibit proinflammatory cytokine release and inflammatory cytokine cascades, with results that can alleviate deleterious symptoms caused by inflammatory cytokine cascades. It has also been discovered that the A box is a weak agonist of inflammatory cytokine release, and competitively inhibits the proinflammatory activity of the B box and of HMG1.

As used herein, an “HMG polypeptide” or an “HMG protein” is a substantially pure, or substantially pure and isolated polypeptide that has been separated from components that naturally accompany it, or a recombinantly produced polypeptide having the same amino acid sequence, and increases inflammation, and/or increases release of a proinflammatory cytokine from a cell, and/or increases the activity of the inflammatory cytokine cascade. In one embodiment, the HMG polypeptide has one of the above biological activities. In another embodiment, the HMG polypeptide has two of the above biological activities. In a third embodiment, the HMG polypeptide has all three of the above biological activities.

Preferably, the HMG polypeptide is a mammalian HMG polypeptide, for example, a human HMG1 polypeptide. Preferably, the HMG polypeptide has at least 60%, more preferably, at least 70%, 75%, 80%, 85%, or 90%, and most preferably at least 95% sequence identity to a sequence selected from SEQ ID NO:1, SEQ ID NO:2, SEQ ID NO:3, or SEQ ID NO:18, as determined using the BLAST program and parameters described herein. Examples of an HMG polypeptide include a polypeptid comprising or consisting of the sequence of SEQ ID NO:1, SEQ ID NO:2, SEQ ID NO:3, or SEQ ID NO:18. Preferably, the HMG polypeptide contains a B box DNA binding domain and/or an A box DNA binding domain, and/or an acidic carboxyl terminus as described herein. Other examples of HMG polypeptides are described in GenBank Accession Numbers AAA64970, AAB08987, P07155, AAA20508, S29857, P09429, NP_{—}002119, CAA31110, the entire teachings of which are incorporated herein by reference. Additional examples of HMG polypeptides include, but are not limited to mammalian HMG1, HMG2, HMG-2A, HMG14, HMG17, HMG I and HMGY; nonmammalian HMG T1 and HMG T2 (rainbow trout), HMG-X (Xenopus), HMG D/Z (Drosophila), yeast polypeptides NHP10 protein (HMG protein homolog NHP 1) and non-histone chromosomal protein; HMG 1/2 like protein (wheat, maize, soybean); upstream binding factor (UBF-1), single-strand recognition protein (SSRP) or structure-specific recognition protein; the HMG homolog TDP-1; mammalian sex-determining region Y protein (SRY, testis-determining factor); fungal proteins: mat-1, ste 11 and Mc 1; SOX 14 (as well as SOX 1-3, 6, 8, 10, 12 and 21); lymphoid specific factor (LEF-1); T-cell specific transcription factor (TCF-1); and SP100-HMG nuclear autoantigen.

As used herein, an “HMG A box” also referred to herein as an “A box” is a substantially pure, or substantially pure and isolated polypeptide that has been separated from components that naturally accompany it, and consists of an amino acid sequence that is less than a full length HMG polypeptide and which has one or more of the following biological activities: inhibiting inflammation, and/or inhibiting release of a proinflammatory cytokine from a cell, and/or decreasing the activity of the inflammatory cytokine cascade. In one embodiment, the HMG A box polypeptide has one of the above biological activities. In another embodiment, the HMG A box polypeptide has two of the above biological activities. In a third embodiment, the HMG A box polypeptide has all three of the above biological activities. Preferably, the HMG A box has no more than 10%, 20%, 25%, 30%, 40%, 50%, 60%, 70%, 80%, or 90% of the biological activity of full length HMG. In one embodiment, the HMG A box amino acid consists of the sequence of SEQ ID NO:4 or SEQ ID NO:22 or the amino acid sequence in the corresponding region of an HMG protein in a mammal. An HMG A box is also a recombinantly produced polypeptide having the same amino acid sequence as the A box sequences described above. Preferably, the HMG A box is a mammalian HMG A box, for example, a human HMG1 A box. The HMG A box polypeptides of the present invention preferably comprise or consist of the sequence of SEQ ID NO:4 or SEQ ID NO:22 or the amino acid sequence in the corresponding region of an HMG protein in a mammal. An HMG A box often has no more than about 85 amino acids and no fewer than about 4 amino acids. Examples of polypeptides having A box sequences within them include, but are not limited to HMG1, HMG2, HMG4; structure-specific recognition protein (SSRP); PMS1 protein homolog 1; SOX-1, SOX-2, and SOX-14 proteins; and MTT1. The A box sequences in such polypeptides can be determined and isolated using methods described herein, for example, by sequence comparisons to A boxes described herein and testing for biological activity.

The present invention also features non-naturally occurring HMG A boxes. Preferably, a non-naturally occurring HMG A box has at least 60%, more preferably, at least 70%, 75%, 80%, 85%, or 90%, and most preferably at least 95% sequence identity to the sequence of SEQ ID NO:4 or SEQ ID NO:22, as determined using the BLAST program and parameters described herein and one of more of the biological activities of an HMG A box.

The present invention also features A box biologically active fragments. By an “A box fragment that has A box biological activity” or an “A box biologically active fragment” is meant a fragment of an HMG A box that has the activity of an HMG A box, as described herein. For example, the A box fragment can decrease release of a pro-inflammatory cytokine from a vertebrate cell, decrease inflammation, and/or decrease activity of the inflammatory cytokine cascade. A box fragments can be generated using standard molecular biology techniques and assaying the function of the fragment by determining if the fragment, when administered to a cell inhibits release of a proinflammatory cytokine from the cell, for example using methods described herein. A box biologically active fragments can be used in the methods described herein in which full length A box polypeptides are used, for example, inhibiting release of a proinflammatory cytokine from a cell, or treating a patient having a condition characterized by activation of an inflammatory cytokine cascade.

As used herein, an “HMG B box” also referred to herein as a “B box” is a substantially pure, or substantially pure and isolated polypeptide that has been separated from components that naturally accompany it, and consists of an amino acid sequence that is less than a full length HMG polypeptide and has one or more of the following biological activities: increasing inflammation, increasing release of a proinflammatory cytokine from a cell, and or increasing the activity of the inflammatory cytokine cascade. In one embodiment, the HMG B box polypeptide has one of the above biological activities. In another embodiment, the HMG B box polypeptide has two of the above biological activities. In a third embodiment, the HMG B box polypeptide has all three of the above biological activities. Preferably, the HMG B box has at least 25%, 30%, 40%, 50%, 60%, 70%, 80% or 90% of the biological activity of full length HMG. In another embodiment, the HMG B box does not comprise an HMG A box. In another embodiment, the HMG B box is a polypeptide that is about 90%, 80%, 70%, 60%, 50%, 40%, 35%, 30%, 25%, or 20% the length of a full length HMG1 polypeptide. In another embodiment, the HMG box comprises or consists of sequence of SEQ ID NO:5 or SEQ ID NO:20 or the amino acid sequence in the corresponding region of an HMG protein in a mammal, but is still less than the full length HMG polypeptide. An HMG B box polypeptide is also a recombinantly produced polypeptide having the same amino acid sequence as an HMG B box polypeptide described above. Preferably, the HMG B box is a mammalian HMG B box, for example, a human HMG1 B box. An HMG B box often has no more than about 85 amino acids and no fewer than about 4 amino acids. Examples of polypeptides having B box sequences within them include, but are not limited to HMG polypeptides described herein; single-strand recognition protein (SSRP) or structure-specific recognition protein; yeast NHP10 protein (HMG protein homolog NHP 1); the HMG homolog TDP-1; sex-determining region Y protein (testis-determining factor); SOX 14 (as well as SOX 1-3, 6, 8, 10, 12 and 21); lymphoid specific factor (LEF-1); and T-cell specific transcription factor (TCF-1). The B box sequences in such polypeptides can be determined and isolated using methods described herein, for example, by sequence comparisons to B boxes described herein and testing for biological activity.

The present invention also includes non-naturally occurring HMG B box polypeptides. Preferably, a non-naturally occurring HMG B box polypeptide has at least 60%, more preferably, at least 70%, 75%, 80%, 85%, or 90%, and most preferably at least 95% sequence identity to the sequence of SEQ ID NO:5 or SEQ ID NO:20, as determined using the BLAST program and parameters described herein. Preferably, the HMG B box consists of the sequence of SEQ ID NO:5 or SEQ ID NO:20 or the amino acid sequence in the corresponding region of an HMG protein in a mammal.

In other embodiments, the present invention is directed to a polypeptide comprising a vertebrate HMG B box or a fragment thereof that has B box biological activity, or a non-naturally occurring HMG B box but not comprising a full length HMG. By a “B Box fragment that has B box biological activity” or a “B box biologically active fragment” is meant a fragment of an HMG B box that has the activity of an HMG B box. For example, the B box fragment can induce release of a proinflammatory cytokine from a vertebrate cell or increase inflammation, or induce the inflammatory cytokine cascade. An example of such a B box fragment is the fragment comprising the first 20 amino acids of the HMG1 B box (SEQ ID NO:16 or SEQ ID NO:23), as described herein. B box fragments can be generated using standard molecular biology techniques and assaying the function of the fragment by determining if the fragment, when administered to a cell increase release of a proinflammatory cytokine from the cell, compared to a suitable control, for example, using methods described herein.

As used herein, a “cytokine” is a soluble protein or peptide which is naturally produced by mammalian cells and which acts in vivo as a humoral regulator at micro- to picomolar concentrations. Cytokines can, either under normal or pathological conditions, modulate the functional activities of individual cells and tissues. A proinflammatory cytokine is a cytokine that is capable of causing any of the following physiological reactions associated with inflammation: vasodialation, hyperemia, increased permeability of vessels with associated edema, accumulation of granulocytes and mononuclear phagocytes, or deposition of fibrin. In some cases, the proinflammatory cytokine can also cause apoptosis, such as in chronic heart failure, where TNF has been shown to stimulate cardiomyocyte apoptosis (Pulkki, Ann. Med. 29:339-343, 1997; and Tsutsui et al., Immunol. Rev. 174:192-209, 2000).

Nonlimiting examples of proinflammatory cytokines are tumor necrosis factor (TNF), interleukin (IL)-1α, IL-1β, IL-6, IL-8, IL-18, interferon γ, HMG-1, platelet-activating factor (PAF), and macrophage migration inhibitory factor (MIF).

Proinflammatory cytokines are to be distinguished from anti-inflammatory cytokines, such as IL-4, IL-10, and IL-13, which are not mediators of inflammation.

In many instances, proinflammatory cytokines are produced in an inflammatory cytokine cascade, defined herein as an in vivo release of at least one proinflammatory cytokine in a mammal, wherein the cytokine release affects a physiological condition of the mammal. Thus, an inflammatory cytokine cascade is inhibited in embodiments of the invention where proinflammatory cytokine release causes a deleterious physiological condition.

HMG A boxes and HMG B boxes, either naturally occurring or non-naturally occurring, include polypeptides that have sequence identity to the HMG A boxes and HMG B boxes described above. As used herein, two polypeptides (or a region of the polypeptides) are substantially homologous or identical when the amino acid sequences are at least about 60%, 70%, 75%, 80%, 85%, 90% or 95% or more homologous or identical. The percent identity of two amino acid sequences (or two nucleic acid sequences) can be determined by aligning the sequences for optimal comparison purposes (e.g., gaps can be introduced in the sequence of a first sequence). The amino acids or nucleotides at corresponding positions are then compared, and the percent identity between the two sequences is a function of the number of identical positions shared by the sequences (i.e., % identity =# of identical positions/total # of positions×100). In certain embodiments, the length of the HMG polypeptide, HMG A box polypeptide, or HMG B box polypeptide aligned for comparison purposes is at least 30%, preferably, at least 40%, more preferably, at least 60%, and even more preferably, at least 70%, 80%, 90%, or 100% of the length of the reference sequence, for example, those sequence provided in FIGS. 12A-12E, and SEQ ID NOS:18, 20, and 22. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA, 90:5873-5877 (1993). Such an algorithm is incorporated into the BLASTN and BLASTX programs (version 2.2) as described in Schaffer et al., Nucleic Acids Res., 29:2994-3005 (2001). When utilizing BLAST and Gapped BLAST programs, the default parameters of the respective programs (e.g., BLASTN) can be used. See http://www.ncbi.nlm.nih.gov, as available on Apr. 10, 2002. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG (Accelrys) sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci., 10:3-5 (1994); and FASTA described in Pearson and Lipman, Proc. Natl. Acad. Sci USA, 85:2444-8 (1988).

In another embodiment, the percent identity between two amino acid sequences can be accomplished using the GAP program in the GCG software package (available at http://www.accelrys.com, as available on Aug. 31, 2001) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (available at http://www.cgc.com), using a gap weight of 50 and a length weight of 3.

A Box Polypeptides and Biologically Active Fragments Thereof

As described above, the present invention is directed to a polypeptide composition comprising a vertebrate HMG A box, or a biologically active fragment thereof which can inhibit release of a proinflammatory cytokine from a vertebrate cell treated with HMG, or which can be used to treat a condition characterized by activation of an inflammatory cytokine cascade.

When referring to the effect of any of the compositions or methods of the invention on the release of proinflammatory cytokines, the use of the terms “inhibit” or “decrease” encompasses at least a small but measurable reduction in proinflammatory cytokine release. In preferred embodiments, the release of the proinflammatory cytokine is inhibited by at least 20% over non-treated controls; in more preferred embodiments, the inhibition is at least 50%; in still more preferred embodiments, the inhibition is at least 70%, and in the most preferred embodiments, the inhibition is at least 80%. Such reductions in proinflammatory cytokine release are capable of reducing the deleterious effects of an inflammatory cytokine cascade in in vivo embodiments.

Because all vertebrate HMG A boxes show a high degree of sequence conservation (see, for example, FIG. 13 for an amino acids sequence comparison of rat, mouse, and human HMG polypeptides), it is believed that any vertebrate HMG A box can inhibit release of a proinflammatory cytokine from a vertebrate cell treated with HMG. Therefore, any vertebrate HMG A box is within the scope of the invention. Preferably, the HMG A box is a mammalian HMG A box, for example, a mammalian HMG1 A box, such as a human HMG1 A box provided herein as SEQ ID NO:4 or SEQ ID NO:22. Also included in the present invention are fragments of the HMG1 A box having HMG A box biological activity, as described herein.

It would also be recognized by the skilled artisan that non-naturally occurring HMG A boxes (or biologically active fragments thereof) can be created without undue experimentation, which would inhibit release of a proinflammatory cytokine from a vertebrate cell treated with a vertebrate HMG. These non-naturally occurring functional A boxes can be created by aligning amino acid sequences of HMG A boxes from different sources, and making one or more substitutions in one of the sequences at amino acid positions where the A boxes differ. The substitutions are preferably made using the same amino acid residue that occurs in the compared A box. Alternatively, a conservative substitution is made from either of the residues.

Conservative amino acid substitutions refer to the interchangeability of residues having similar side chains. Conservatively substituted amino acids can be grouped according to the chemical properties of their side chains. For example, one grouping of amino acids includes those amino acids have neutral and hydrophobic side chains (a, v, l, i, p, w, f, and m); another grouping is those amino acids having neutral and polar side chains (g, s, t, y, c, n, and q); another grouping is those amino acids having basic side chains (k, r, and h); another grouping is those amino acids having acidic side chains (d and e); another grouping is those amino acids having aliphatic side chains (g, a, v, l, and i); another grouping is those amino acids having aliphatic-hydroxyl side chains (s and t); another grouping is those amino acids having amine-containing side chains (n, q, k, r, and h); another grouping is those amino acids having aromatic side chains (f, y, and w); and another grouping is those amino acids having sulfur-containing side chains c and m). Preferred conservative amino acid substitutions groups are: r-k; e-d, y-f, l-m; v-i, and q-h.

While a conservative amino acid substitution would be expected to preserve the biological activity of an HMG A box polypeptide, the following is one example of how non-naturally occurring A box polypeptides can be made by comparing the human HMG1 A box (SEQ ID NO:4) with residues 32 to 85 of SEQ ID NO:3 of the human HMG2 A box (SEQ ID NO:17).

HMG1 pdasvnfsef skkcserwkt msakekgkfe dmakadkary eremktyipp kget

HMG2 pdssvnfaef skkcserwkt msakekskfe dmaksdkary dremknyvpp kgdk

A non-naturally occurring HMG A box can be created by, for example, by substituting the alanine (a) residue at the third position in the HMG1 A box with the serine (s) residue that occurs at the third position of the HMG2 A box. The skilled artisan would know that the substitution would provide a functional non-naturally occurring A box because the s residue functions at that position in the HMG2 A box. Alternatively, the third position of the HMG1 A box can be substituted with any amino acid that is conservative to alanine or serine, such as glycine (g), threonine (t), valine (v) or leucine (l). The skilled artisan would recognize that these conservative substitutions would be expected to result in a functional A box because A boxes are not invariant at the third position, so a conservative substitution would provide an adequate structural substitute for an amino acid that is naturally occurring at that position.

Following the above method, a great many non-naturally occurring HMG A boxes could be created without undue experimentation which would be expected to be functional, and the functionality of any particular non-naturally occurring HMG A box could be predicted with adequate accuracy. In any event, the functionality of any non-naturally occurring HMG A box could be determined without undue experimentation by simply adding it to cells along with an HMG, and determine whether the A box inhibits release of a proinflammatory cytokine by the cells, using, for example, methods described herein.

The cell from which the A box or an A box biologically active fragment will inhibit the release of HMG-induced proinflammatory cytokines can be any cell that can be induced to produce a proinflammatory cytokine. In preferred embodiments, the cell is an immune cell, for example, a macrophage, a monocyte, or a neutrophil. In the most preferred embodiment, the cell is a macrophage.

Polypeptides comprising an A box or A box biologically active fragment that can inhibit the production of any single proinflammatory cytokine, now known or later discovered, are within the scope of the present invention. Preferably, the antibodies can inhibit the production of TNF, IL-1β, or IL-6. Most preferably, the antibodies can inhibit the production of any proinflammatory cytokines produced by the vertebrate cell.

The present invention is also directed to a composition comprising any of the above-described polypeptides, in a pharmaceutically acceptable excipient. In these embodiments, the composition can inhibit a condition characterized by activation of an inflammatory cytokine cascade. The condition can be one where the inflammatory cytokine cascade causes a systemic reaction, such as with endotoxic shock. Alternatively, the condition can be mediated by a localized inflammatory cytokine cascade, as in rheumatoid arthritis. Nonlimiting examples of conditions which can be usefully treated using the present invention include those conditions enumerated in the background section of this specification. Preferably, the condition is appendicitis, peptic, gastric or duodenal ulcers, peritonitis, pancreatitis, ulcerative, pseudomembranous, acute or ischemic colitis, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopicsilicovolcanoconiosis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, Type I diabetes, ankylosing spondylitis, Berger's disease, Retier's syndrome, or Hodgkins disease. In more preferred embodiments, the condition is appendicitis, peptic, gastric or duodenal ulcers, peritonitis, pancreatitis, ulcerative, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, coeliac disease, congestive heart failure, adult respiratory distress syndrome, cerebral infarction, cerebral embolism, spinal cord injury, paralysis, allograft rejection or graft-versus-host disease. In the most preferred embodiments, the condition is endotoxic shock or allograft rejection. Where the condition is allograft rejection, the composition may advantageously also include an immunosuppressant that is used to inhibit allograft rejection, such as cyclosporin.

The excipient included with the polypeptide in these compositions is chosen based on the expected route of administration of the composition in therapeutic applications. The route of administration of the composition depends on the condition to be treated. For example, intravenous injection may be preferred for treatment of a systemic disorder such as endotoxic shock, and oral administration may be preferred to treat a gastrointestinal disorder such as a gastric ulcer. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. Thus, depending on the condition, the antibody composition can be administered orally, parenterally, intranasally, vaginally, rectally, lingually, sublingually, bucally, intrabuccaly and transdermally to the patient.

Accordingly, compositions designed for oral, lingual, sublingual, buccal and intrabuccal administration can be made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used.

The compositions of the present invention can easily be administered parenterally such as, for example, by intravenous, intramuscular, intrathecal or subcutaneous injection. Parenteral administration can be accomplished by incorporating the antibody compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic. Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the antibody composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the agonist prepared by well-known methods to be administered, for example, as a nasal spray, nasal drop, suspension, gel, ointment, cream or powder. Administration of the composition may also take place using a nasal tampon or nasal sponge.

The polypeptide compositions described herein can also include an antagonist of an early sepsis mediator. As used herein, an early sepsis mediator is a proinflammatory cytokine that is released from cells soon (i.e., within 30-60 min.) after induction of an inflammatory cytokine cascade (e.g., exposure to LPS). Nonlimiting examples of these cytokines are TNF, IL-1α, IL-1β, IL-6, PAF, and MIF. Also included as early sepsis mediators are receptors for these cytokines (for example, tumor necrosis factor receptor type 1) and enzymes required for production of these cytokines, for example, interleukin-1β converting enzyme). Antagonists of any early sepsis mediator, now known or later discovered, can be useful for these embodiments by further inhibiting an inflammatory cytokine cascade.

Nonlimiting examples of antagonists of early sepsis mediators are antisense compounds that bind to the mRNA of the early sepsis mediator, preventing its expression (see, e.g., Ojwang et al., Biochemistry 36:6033-6045, 1997; Pampfer et al., Biol. Reprod. 52:1316-1326, 1995; U.S. Pat. No. 6,228,642; Yahata et al., Antisense Nucleic Acid Drug Dev. 6:55-61, 1996; and Taylor et al., Antisense Nucleic Acid Drug Dev. 8:199-205, 1998), ribozymes that specifically cleave the mRNA of the early sepsis mediator (see, e.g., Leavitt et al., Antisense Nucleic Acid Drug Dev. 10:409-414, 2000; Kisich et al., 1999; and Hendrix et al., Biochem. J. 314 (Pt. 2):655-661, 1996), and antibodies that bind to the early sepsis mediator and inhibit their action (see, e.g., Kam and Targan, Expert Opin. Pharmacother. 1:615-622, 2000; Nagahira et al., J. Immunol. Methods 222, 83-92, 1999; Lavine et al., J. Cereb. Blood Flow Metab. 18:52-58, 1998; and Holmes et al., Hybridoma 19: 363-367, 2000). Any antagonist of an early sepsis mediator, now known or later discovered, is envisioned as within the scope of the invention. The skilled artisan can determine the amount of early sepsis mediator to use in these compositions for inhibiting any particular inflammatory cytokine cascade without undue experimentation with routine dose-response studies.

B Box Polypeptides, Biologically Active Fragments thereof, and Antibodies Thereto

As described above, the present invention is directed to a polypeptide composition comprising a vertebrate HMG B box, or a biologically active fragment thereof which can increase release of a proinflammatory cytokine from a vertebrate cell treated with HMG.

When referring to the effect of any of the compositions or methods of the invention on the release of proinflammatory cytokines, the use of the term “increase” encompasses at least a small but measurable rise in proinflammatory cytokine release. In preferred embodiments, the release of the proinflammatory cytokine is increased by at least 1.5-fold, at least 2-fold, at least 5-fold, or at least 10-fold over non-treated controls. Such increases in proinflammatory cytokine release are capable of increasing the effects of an inflammatory cytokine cascade in in vivo embodiments. Such polypeptides can also be used to induce weight loss and/or treat obesity.

Because all HMG B boxes show a high degree of sequence conservation (see, for example, FIG. 13 for an amino acids sequence comparison of rat, mouse, and human HMG polypeptides), it is believed that functional non-naturally occurring HMG B boxes can be created without undue experimentation by making one or more conservative amino acid substitutions, or by comparing naturally occurring vertebrate B boxes from different sources and substituting analogous amino acids, as was discussed above with respect to the creation of functional non-naturally occurring A boxes. In particularly preferred embodiments, the B box comprises SEQ ID NO:5 or SEQ ID NO: 20), which are the sequences (two different lengths) of human HMG1 B box, or is a fragment of an HMG B box that has B box biological activity. For example, a 20 amino acid sequence contained within SEQ ID NO: 20 contributes to the function of the B box. This 20 amino acid B-box fragment has the following amino acid sequence: fkdpnapkrl psafflfcse (SEQ ID NO:16). Another example of an HMG B box biologically active fragment consists of amino acids 1-20 of SEQ ID NO:5 (napkrppsaf flfcseyrpk; SEQ ID NO: 23).

The invention is also directed to a purified preparation of antibodies that specifically bind to a vertebrate high mobility group protein (HMG) B box, but do not specifically bind to non-B box epitopes of HMG1. In these embodiments, the antibodies can inhibit a biological activity of a B box polypeptide, for example, the release of a proinflammatory cytokine from a vertebrate cell induced by HMG.

To make antibodies specific to the HMG B box or fragments thereof, or cells expressing the B box or epitope-bearing fragments can be used as an immunogen to produce antibodies immunospecific for the immunogen. “Antibodies” as used herein includes monoclonal and polyclonal antibodies, chimeric, single chain, simianized antibodies and humanized antibodies, as well as Fab fragments, including the products of an Fab immunoglobulin expression library.

Because all vertebrate HMG B boxes show a high degree of sequence conservation, it is believed that any vertebrate HMG B box can induce release of a proinflammatory cytokine from a vertebrate cell. Therefore, antibodies against any vertebrate HMG B box are within the scope of the invention. Preferably, the HMG B box is a mammalian HMG B box, more preferably a mammalian HMG1 B box, most preferably a human HMG1 B box, provided herein as SEQ ID NO:5 or SEQ ID NO:20. Antibodies can also be directed against an HMG B box fragment that has B box biological activity.

Antibodies generated against the B box immunogen can be obtained by administering the B box, a B box fragment, or cells comprising the B box or B box fragment to an animal, preferably a nonhuman, using routine protocols. The polypeptide, such as an antigenically or immunologically equivalent derivative or a fusion protein thereof is used as an antigen to immunize a mouse or other animal such as a rat or chicken. The B box or fragment immunogen can be provided as a fusion protein to provide stability or increase the immunogenicity of the B box or fragment. The immunogen may be associated, for example, by conjugation, with an immunogenic carrier protein, for example, bovine serum albumin (BSA) or keyhole limpet haemocyanin (KLH). Alternatively a multiple antigenic peptide comprising multiple copies of the B box or fragment, may be sufficiently antigenic to improve immunogenicity so as to obviate the use of a carrier. Bispecific antibodies, having two antigen binding domains where each is directed against a different B box epitope, may also be produced by routine methods.

For preparation of monoclonal antibodies, any technique known in the art that provides antibodies produced by continuous cell line cultures can be used. See, e.g., Kohler and Milstein, Nature 256:495-497, 1975; Kozbor et al., Immunology Today 4:72, 1983; and Cole et al., pg. 77-96 in MONOCLONAL ANTIBODIES AND CANCER THERAPY, Alan R. Liss, Inc., 1985.

Techniques for the production of single chain antibodies (U.S. Pat. No. 4,946,778) can be adapted to produce single chain antibodies to the B box or fragments. Also, transgenic mice, or other organisms such as other mammals, may be used to express humanized antibodies.

If the antibody is used therapeutically in in vivo applications, the antibody is preferably modified to make it less immunogenic in the individual. For example, if the individual is human the antibody is preferably “humanized”; where the complementarity determining region(s) of the antibody is transplanted into a human antibody (for example, as described in Jones et al., Nature 321:522-525, 1986; and Tempest et al., Biotechnology 9:266-273, 1991).

Phage display technology can also be utilized to select antibody genes with binding activities towards the polypeptide either from repertoires of PCR amplified v-genes of lymphocytes from humans screened for possessing anti-B box antibodies or from naive libraries (McCafferty et al., Nature 348:552-554, 1990; and Marks, et al., Biotechnology 10:779-783, 1992). The affinity of these antibodies can also be improved by chain shuffling (Clackson et al., Nature 352:624-628, 1991).

When the antibodies are obtained that specifically bind to HMG B box epitopes, they can then be screened without undue experimentation for the ability to inhibit release of a proinflammatory cytokine.

Anti-HMG B box antibodies that can inhibit the production of any single proinflammatory cytokine are within the scope of the present invention. Preferably, the antibodies can inhibit the production of TNF, IL-1β, or IL-6. Most preferably, the antibodies can inhibit the production of any proinflammatory cytokines produced by the vertebrate cell.

For methods of inhibiting release of a proinflammatory cytokine from a cell or treating a condition characterized by activation of an inflammatory cytokine cascade using antibodies to the HMG B box or a biologically active fragment thereof, the cell can be any cell that can be induced to produce a proinflammatory cytokine. In preferred embodiments, the cell is an immune cell, for example, macrophages, monocytes, or neutrophils. In the most preferred embodiments, the cell is a macrophage.

In other embodiments, the present invention is directed to a composition comprising the antibody preparations described above, in a pharmaceutically acceptable excipient. In these embodiments, the compositions can inhibit a condition characterized by the activation of an inflammatory cytokine cascade. Conditions that can be treated with these compositions have been previously enumerated.

The antibody compositions described above can also include an antagonist of an early sepsis mediator, as previously described.

The B box polypeptides and biologically active fragments thereof described in these embodiments can be used to induce inflammatory cytokines in the appropriate isolated cells in vitro, or ex vivo, or as a treatment in vivo. In any of these treatments, the polypeptide or fragment can be administered by providing a DNA or RNA vector encoding the B box or B box fragment, with the appropriate control sequences operably linked to the encoded B box or B box fragment, so that the B box or B box fragment is synthesized in the treated cell or patient. In vivo applications include the use of the B box polypeptides or B box fragment polypeptides or vectors as a weight loss treatment. See WO 00/47104 (the entire teachings of which are incorporated herein by reference), demonstrating that treatment with HMG1 induces weight loss. Since the HMG B box has the activity of the HMG protein, the B box would also be expected to induce weight loss. HMG B box fragments that have the function of the B box would also be expected to induce weight loss.

In further embodiments, the present invention is also directed to a method of inhibiting the release of a proinflammatory cytokine from a mammalian cell. The method comprises treating the cell with any of the HMG A box compositions or any of the HMG B box or HMG B box biologically active fragment antibody compositions discussed above.

It is believed that this method would be useful for inhibiting the cytokine release from any mammalian cell that produces the proinflammatory cytokine. However, in preferred embodiments, the cell is a macrophage, because macrophage production of proinflammatory cytokines is associated with several important diseases.

It is believed that this method is useful for the inhibition of any proinflammatory cytokine produced by mammalian cells. In preferred embodiments, the proinflammatory cytokine is TNF, IL-1α, IL-1β, MIF or IL-6, because those proinflammatory cytokines are particularly important mediators of disease.

The method of these embodiments is useful for in vitro applications, such as in studies for determining biological characteristics of proinflammatory cytokine production in cells. However, the preferred embodiments are in vivo therapeutic applications, where the cells are in a patient suffering from, or at risk for, a condition characterized by activation of an inflammatory cytokine cascade.

These in vivo embodiments are believed to be useful for any condition that is mediated by an inflammatory cytokine cascade, including any of those that have been previously enumerated. Preferred conditions include appendicitis, peptic, gastric or duodenal ulcers, peritonitis, pancreatitis, ulcerative, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, cerebral infarction, cerebral embolism, spinal cord injury, paralysis, allograft rejection or graft-versus-host disease. In the most preferred embodiments, the condition is endotoxic shock or allograft rejection. Where the condition is allograft rejection, the composition may advantageously also include an immunosuppressant that is used to inhibit allograft rejection, such as cyclosporin.

These methods can also usefully include the administration of an antagonist of an early sepsis mediator. The nature of these antagonists has been previously discussed.

In still other embodiments, the present invention is directed to a method of treating a condition in a patient characterized by activation of an inflammatory cytokine cascade. The method comprises administering to the patient with any of the HMG A box compositions (including non-naturally occurring A box polypeptides and A box biologically active fragments) or any of the HMG B box or B box biologically active fragment antibody compositions (including non-naturally occurring B box polypeptides or biologically active fragments thereof) discussed above. This method would be expected to be useful for any condition that is mediated by an inflammatory cytokine cascade, including any of those that have been previously enumerated. As with previously described in vivo methods, preferred conditions include appendicitis, peptic, gastric or duodenal ulcers, peritonitis, pancreatitis, ulcerative, pseudomembranous, acute or ischemic colitis, hepatitis, Crohn's disease, asthma, allergy, anaphylactic shock, organ ischemia, reperfusion injury, organ necrosis, hay fever, sepsis, septicemia, endotoxic shock, cachexia, septic abortion, disseminated bacteremia, burns, Alzheimer's disease, cerebral infarction, cerebral embolism, spinal cord injury, paralysis, allograft rejection or graft-versus-host disease. In the most preferred embodiments, the condition is endotoxic shock or allograft rejection. Where the condition is allograft rejection, the composition may advantageously also include an immunosuppressant that is used to inhibit allograft rejection, such as cyclosporin.

These methods can also usefully include the administration of an antagonist of an early sepsis mediator. The nature of these antagonists has been previously discussed.

In other embodiments, the present invention is directed to methods of stimulating the release of a proinflammatory cytokine from a cell. The method comprises treating the cell with any of the B box polypeptides or biologically active B box fragment polypeptides, for example, the sequence of SEQ ID NO:5, SEQ ID NO:20, SEQ ID NO:16, or SEQ ID NO:23, as described herein (including non-naturally occurring B box polypeptides and fragments). This method is useful for in vitro applications, for example, for studying the effect of proinflammatory cytokine production on the biology of the producing cell. The method is also useful for in vivo applications, for example, in effecting weight loss or treating obesity in a patient, as previously discussed.

Thus, in additional embodiments, the present invention is directed to a method for effecting weight loss or treating obesity in a patient. The method comprises administering to the patient an effective amount of any of the B box polypeptides or B box fragment polypeptides described herein (including non-naturally occurring B box polypeptides and fragments), in a pharmaceutically acceptable excipient.

Screening for Modulators of the Release of Proinflammatory Cytokines from Cells

The present invention is also directed to a method of determining whether a compound (test compound) inhibits inflammation and/or an inflammatory response. The method comprises combining the compound with (a) a cell that releases a proinflammatory cytokine when exposed to a vertebrate HMG B box or a biologically active fragment thereof, and (b) the HMG B box or a biologically active fragment thereof, then determining whether the compound inhibits the release of the proinflammatory cytokine from the cell, compared to a suitable control. A compound that inhibits the release of the proinflammatory cytokine in this assay is a compound that can be used to treat inflammation and/or an inflammatory response. The HMG B box or biologically active HMG B box fragment can be endogenous to the cell or can be introduced into the cell using standard recombinant molecular biology techniques.

Any cell that releases a proinflammatory cytokine in response to exposure to a vertebrate HMG B box or biologically active fragment thereof in the absence of a test compound would be expected to be useful for this invention. It is envisioned that the cell that is selected would be important in the etiology of the condition to be treated with the inhibitory compound that is being tested. For many conditions, it is expected that the preferred cell is a human macrophage.

Any method for determining whether the compound inhibits the release of the proinflammatory cytokine from the cell would be useful for these embodiments. It is envisioned that the preferred methods are the direct measurement of the proinflammatory cytokine, for example, with any of a number of commercially available ELISA assays. However, in some embodiments, the measurement of the inflammatory effect of released cytokines may be preferable, particularly when there are several proinflammatory cytokines produced by the test cell. As previously discussed, for many important disorders, the predominant proinflammatory cytokines are TNF, IL-1α, IL-1β, MIF or IL-6; particularly TNF.

The present invention also features a method of determining whether a compound increases an inflammatory response and/or inflammation. The method comprises combining the compound (test compound) with (a) a cell that releases a proinflammatory cytokine when exposed to a vertebrate HMG A box or a biologically active fragment thereof, and (b) the HMG A box or biologically active fragment, then determining whether the compound increases the release of the proinflammatory cytokine from the cell, compared to a suitable control. A compound that decreases the release of the proinflammatory cytokine in this assay is a compound that can be used to increase an inflammatory response and/or inflammation. The HMG A box or HMG A box biologically active fragment can be endogenous to the cell or can be introduced into the cell using standard recombinant molecular biology techniques.

Similar to the cell types useful for identifying inhibitors of inflammation, described above, any cell in which release of a proinflammatory cytokine is normally inhibited in response to exposure to a vertebrate HMG A box or a biologically active fragment thereof in the absence of any test compound would be expected to be useful for this invention. It is envisioned that the cell that is selected would be important in the etiology of the condition to be treated with the inhibitory compound that is being tested. For many conditions, it is expected that the preferred cell is a human macrophage.

Any method for determining whether the compound increases the release of the proinflammatory cytokine from the cell would be useful for these embodiments. It is envisioned that the preferred methods are the direct measurement of the proinflammatory cytokine, for example, with any of a number of commercially available ELISA assays. However, in some embodiments, the measurement of the inflammatory effect of released cytokines may be preferable, particularly when there are several proinflammatory cytokines produced by the test cell. As previously discussed, for many important disorders, the predominant proinflammatory cytokines are TNF, IL-1α, IL-1β, MIF or IL-6; particularly TNF.

Preferred embodiments of the invention are described in the following examples. Other embodiments within the scope of the invention will be apparent to one skilled in the art from consideration of the specification or practice of the invention as disclosed herein. It is intended that the specification, together with the examples and claims, be considered exemplary only.

### EXAMPLE 1

### Materials and Methods

Cloning of HMG1 and Production of HMG1 Mutants

The following methods were used to prepare clones and mutants of human HMG1. Recombinant full length human HMG1 (651 base pairs; GenBank Accession Number U51677) was cloned by PCR amplification from a human brain Quick-Clone cDNA preparation (Clontech, Palo Alto, Calif.) using the following primers; forward primer: 5′ GATGGGCAAAGGAGATCCTAAG 3′ (SEQ ID NO:6) and reverse primer: 5′ GCGGCCGCTTATTCATCATCATCATCTTC 3′ (SEQ ID NO:7). Human HMG1 mutants were cloned and purified as follows. A truncated form of human HMG1 was cloned by PCR amplification from a Human Brain Quick-Clone cDNA preparation (Clontech, Palo Alto, Calif.). The primers used were (forward and reverse, respectively):

| | | |
|---|---|---|
| Carboxy terminus mutant (557 bp): | 5′GATGGGCAAAGGAGATCCTAAG 3′ and | (SEQ ID NO:8) |
| | 5′GCGGCCGC TCACTTGCTTTTTTCAGCCTTGAC 3′. | (SEQ ID NO:9) |
| Amino terminus + B box mutant (486 bp): | 5′GAGCATAAGAAGAAGCACCCA 3′ and | (SEQ ID NO:10) |
| | 5′GCGGCCGC TCACTTGCTTTTTTCAGCCTTGAC 3′. | (SEQ ID NO:11) |
| B box mutant (233 bp): | 5′AAGTTCAAGGATCCCAATGCAAAG 3′ and | (SEQ ID NO:12) |
| | 5′GCGGCCGCTCAATATGCAGCTATATCCTTTTC 3′. | (SEQ ID NO:13) |
| Amino terminus + A box mutant (261 bp): | 5′GATGGGCAAAGGAGATCCTAAG 3′ and | (SEQ ID NO:13) |
| | 5′TCACTTTTTTGTCTCCCCTTTGGG 3′. | (SEQ ID NO:14) |

A stop codon was added to each mutant to ensure the accuracy of protein size. PCR products were subcloned into pCRII-TOPO vector EcoRI sites using the TA cloning method per manufacturer's instruction (Invitrogen, Carlsbad, Calif.). After amplification, the PCR product was digested with EcoRI and subcloned onto expression vector with a GST tag pGEX (Pharmacia); correct orientation and positive clones were confirmed by DNA sequencing on both strands. The recombinant plasmids were transformed into protease deficient E. Coli strains BL21 or BL21(DE3)plysS (Novagen, Madison, Wis.) and fusion protein expression was induced by isopropyl-D-thiogalactopyranoside (IPTG). Recombinant proteins were obtained using affinity purification with the glutathione Sepharose resin column (Pharmacia).

The HMG mutants generated as described above have the following amino acid sequences:

| | |
|---|---|
| Wild type HMG1: | |
| MGKGDPKKPTGKMSSYAFFVQTCREEHKKKHPDASVNFSEF | (SEQ ID NO:18) |
| SKKCSERWKTMSAKEKGKFEDMAKADKARYEREMKTYIPPKGETKKKFKDPN | |
| APKRLPSAFFLFCSEYRPKIKGEHPGLSIGDVAKKLGEMWNNTAADDKQPYEK | |
| KAAKLKEKYEKDIAAYRAKGKPDAAKKGVVKAEKSKKKKEEEEDEEDEEDEE | |
| EEEDEEDEEDEEEDDDDE | |
| Carboxy terminus mutant: | |
| MGKGDPKKPTGKMSSYAFFVQTCREEHKKKHPDAS | (SEQ ID NO:19) |
| VNFSEFSKKCSERWKTMSAKEKGKFEDMAKADKARYEREMKTYIPPKGETKK | |
| KFKDPNAPKRLPSAFFLFCSEYRPKIKGEHPGLSIGDVAKKLGEMWNNTAADDK | |
| QPYEKKAAKLKEKYEKDIAAYRAKGKPDAAKKGVVKAEKSK | |
| B Box mutant: | |
| FKDPNAPKRLPSAFFLFCSEYRPKIKGEHPGLSIGDVAKKLGEM | (SEQ ID NO:20) |
| WNNTAADDKQPYEKKAAKLKEKYEKDIAAY | |
| Amino terminus+A Box mutant: | |
| MGKGDPKKPTGKMSSYAFFVQTCREEHKKK | (SEQ ID NO:21) |
| HPDASVNFSEFSKKCSERWKTMSAKEKGKFEDMAKADKARYEREMKTYIPPK | |
| GET, | |
| wherein the A box consists of the sequence PTGKMSSYAFF | (SEQ ID NO:22) |
| VQTCREEHKKKHPDASVNFSEFSKKCSERWKTMSAKEKGKFEDMAKADKAR | |
| YEREMKTYIPPKGET | |

A polypeptide generated from a GST vector lacking HMG1 protein was included as a control (containing a GST tag only). To inactive the bacterial DNA that bound to the wild type HMG1 and some of the mutants (carboxy terminus and B box), DNase I (Life Technologies), for carboxy terminus and B box mutants, or benzonase nuclease (Novagen, Madison, Wis.), for wild type HMG1, was added at about 20 units/ml bacteria lysate. Degradation of DNA was verified by ethidium bromide staining of the agarose gel containing HMG1 proteins before and after the treatment. The protein eluates were passed over a polymyxin B column (Pierce, Rockford, Ill.) to remove any contaminating LPS, and dialyzed extensively against phosphate buffered saline to remove excess reduced glutathione. The preparations were then lyophilized and redissolved in sterile water before use. LPS levels were less than 60 pg/μg protein for all the mutants and 300 pg/μg for wild type HMG-1 as measured by Limulus amebocyte lysate assay (Bio Whittaker Inc., Walkersville, Md.). The integrity of protein was verified by SDS-PAGE. Recombinant rat HMG1 (Wang et al., Science 285:248-251, 1999) was used in some experiments since it does not have degraded fragments as observed in purified human HMG1.

Peptide Synthesis

Peptides were synthesized and HPLC purified at Utah State University Biotechnology Center (Logan, Utah) at 90% purity. Endotoxin was not detectable in the synthetic peptide preparations as measured by Limulus assay.

Cell Culture

Murine macrophage-like RAW 264.7 cells (American Type Culture Collection, Rockville, Md.) were cultured in RPMI 1640 medium (Life Technologies, Grand Island N.Y.) supplemented with 10% fetal bovine serum (Gemini, Catabasas, Calif.), penicillin and streptomycin (Life Technologies) and were used at 90% confluence in serum-free Opti-MEM I medium (Life Technologies, Grand Island, N.Y.). Polymyxin B (Sigma, St. Louis, Mo.) was routinely added at 100-1,000 units/ml to neutralize the activity of any contaminating LPS as previously described; polymyxin B alone did not influence cell viability assessed with trypan blue (Wang et al., supra). Polymyxin B was not used in experiments of synthetic peptide studies.

Measurement of TNF Release from Cells

TNF release was measured by a standard murine fibroblast L929 (ATCC, American Type Culture Collection, Rockville, Md.) cytotoxicity bioassay (Bianchi et al., supra) with the minimum detectable concentration of 30 pg/ml. Recombinant mouse TNF was obtained from R&D system Inc., (Minneapolis, Minn.). Murine fibroblast L929 cells (ATCC) were cultured in DMEM (Life Technologies, Grand Island, N.Y.) supplemented with 10% fetal bovine serum (Gemini, Catabasas, Calif.), penicillin (50 units/ml) and streptomycin (50 μg/ml) (Life Technologies) in a humidified incubator with 5% CO₂.

Antibody Production

Polyclonal antibodies against HMG1 B box were raised in rabbits (Cocalico Biologicals, Inc., Reamstown, Pa.) and assayed for titer by immunoblotting. IgG was purified from anti-HMG1 antiserum using Protein A agarose according to manufacturer's instructions (Pierce, Rockford, Ill.). Anti-HMG1 B box antibodies were affinity purified by using cyanogen bromide activated Sepharose beads (Cocalico Biological, Inc.). Non-immune rabbit IgG was purchased from Sigma (St. Louis, Mo.). Antibodies detected full length HMG1 and B box in immunoassay, but did not cross react with TNF, IL-1 and IL-6.

Labeling of HMG1 with Na-¹²⁵I and Cell Surface Binding

Purified HMG1 protein (10 μg) was radiolabeled with 0.2 mCi of carrier-free ¹²⁵I (NEN Life Science products Inc., Boston, Mass.) using Iodo-beads (Pierce, Rockford, Ill.) according to the manufacturer's instructions. ¹²⁵I-HMG1 protein was separated from un-reacted ¹²⁵I by gel chromatography columns (P6 Micro Bio-Spin Chromatography Columns, Bio-Rad Laboratories, Hercules, Calif.) previously equilibrated with 300 mM sodium chloride, 17.5 mM sodium citrate, pH 7.0 and 0.1% bovine serum albumin (BSA). The specific activity of the eluted HMG1 was about 2.8×10⁶ cpm/μg protein. Cell surface binding studies were performed as previously described (Yang et al., Am. J. Physiol. 275:C675-C683, 1998). RAW 264.7 cells were plated on 24-well dishes and grown to confluence. Cells were washed twice with ice-cold PBS containing 0.1% BSA and binding was carried out at 4° C. for 2 hours with 0.5 ml binding buffer containing 120 mM sodium chloride, 1.2 mM magnesium sulfate, 15 mM sodium acetate, 5 mM potassium chloride, 10 mM Tris.HCl, pH 7.4, 0.2% BSA, 5 mM glucose and 25,000 cpm ¹²⁵I-HMG1. At the end of the incubation the supernatants were discarded and the cells were washed three times with 0.5 ml ice-cold PBS with 0.1% BSA and lysed with 0.5 ml of 0.5 N NaOH and 0.1% SDS for 20 minutes at room temperature. The radioactivity in the lysate was then measured using a gamma counter. Specific binding was determined as total binding minus the radioactivity obtained in the presence of an excess amount of unlabeled HMG1 or A box proteins.

Animal Experiments

TNF knock out mice were obtained from Amgen (Thousand Oaks, Calif.) and were on a B6x129 background. Age-matched wild-type B6x129 mice were used as control for the studies. Mice were bred in-house at the University of Florida specific pathogen-free transgenic mouse facility (Gainesville, Fla.) and were used at 6-8 weeks of age.

Male 6-8 week old Balb/c and C3H/HeJ mice were purchased from Harlen Sprague-Dawley (Indianapolis, Ind.) and were allowed to acclimate for 7 days before use in experiments. All animals were housed in the North Shore University Hospital Animal Facility under standard temperature, and a light and dark cycle.

Cecal Ligation and Puncture

Cecal ligation and puncture (CLP) was performed as described previously (Fink and Heard, J. Surg. Res. 49:186-196, 1990; Wichmann et al., Crit. Care Med. 26:2078-2086, 1998; and Remick et al., Shock 4:89-95, 1995). Briefly, Balb/c mice were anesthetized with 75 mg/kg ketamine (Fort Dodge, Fort Dodge, Iowa) and 20 mg/kg of xylazine (Bohringer Ingelheim, St. Joseph, Mo.) intramuscularly. A midline incision was performed, and the cecum was isolated. A 6-0 prolene suture ligature was placed at a level 5.0 mm from the cecal tip away from the ileocecal valve.

The ligated cecal stump was then punctured once with a 22-gauge needle, without direct extrusion of stool. The cecum was then placed back into its normal intra-abdominal position. The abdomen was then closed with a running suture of 6-0 prolene in two layers, peritoneum and fascia separately to prevent leakage of fluid. All animals were resuscitated with a normal saline solution administered sub-cutaneously at 20 ml/kg of body weight. Each mouse received a subcutaneous injection of imipenem (0.5 mg/mouse) (Primaxin, Merck & Co., Inc., West Point, Pa.) 30 minutes after the surgery. Animals were then allowed to recuperate. Mortality was recorded for up to 1 week after the procedure; survivors were followed for 2 weeks to ensure no late mortalities had occurred.

D-galactosamine Sensitized Mice

The D-galactosamine-sensitized model has been described previously (Galanos et al., Proc Natl. Acad. Sci. USA 76: 5939-5943, 1979; and Lehmann et al., J. Exp. Med. 165: 657-663, 1997). Mice were injected intraperitoneally with 20 mg D-galactosamine-HCL (Sigma)/mouse (in 200 μl PBS) and 0.1 or 1 mg of either HMG1 B box or vector protein (in 200 μl PBS). Mortality was recorded daily for up to 72 hours after injection; survivors were followed for 2 weeks, and no later deaths from B box toxicity were observed.

Spleen Bacteria Culture

Fourteen mice received either anti-HMG1 antibody (n=7) or control (n=7) at 24 and 30 hours after CLP, as described herein, and were euthanized for necropsy. Spleen bacteria were recovered as described previously (Villa et al., J. Endotoxin Res. 4:197-204, 1997). Spleens were removed using sterile technique and homogenized in 2 ml PBS. After serial dilutions with PBS, the homogenate was plated as 0.15 ml aliquots on tryptic soy agar plates (Difco, Detroit, Mich.) and CFU were counted after overnight incubation at 37° C.

Statistical Analysis

Data are presented as mean±SEM unless otherwise stated. Differences between groups were determined by two-tailed Student's t-test, one-way ANOVA followed by the least significant difference test or 2 tailed Fisher's Exact Test.

### EXAMPLE 2

### Mapping the HMG1 Domains for Promotion of Cytokine Activity

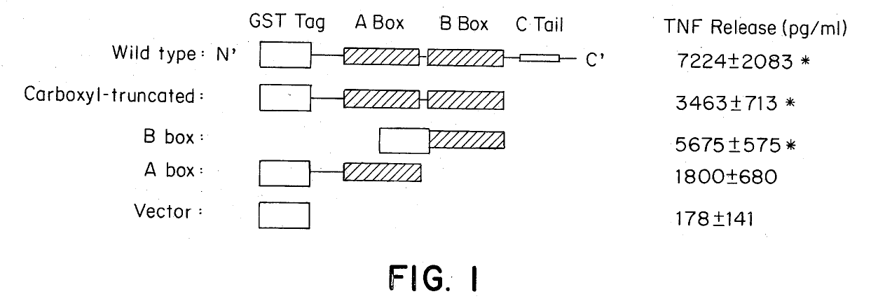

HMG1 has 2 folded DNA binding domains (A and B boxes) and a negatively charged acidic carboxyl tail). To elucidate the structural basis of HMG1 cytokine activity, and to map the inflammatory protein domain, we expressed full length and truncated forms of HMG1 by mutagenesis and screened the purified proteins for stimulating activity in monocyte cultures (FIG. 1). Full length HMG1, a mutant in which the carboxy terminus was deleted, a mutant containing only the B box, and a mutant containing only the A box were generated. These mutants of human HMG1 were made by polymerase chain reaction (PCR) using specific primers as described herein, and the mutant proteins were expressed using a glutathione S-transferase (GST) gene fusion system (Pharmacia Biotech, Piscataway, N.J.) in accordance with the manufacturer's instructions. Briefly, DNA fragments, made by PCR methods, were fused to GST fusion vectors and amplified in E. Coli. The expressed HMG1 protein and HMG1 mutants and were then isolated using GST affinity column.

The effect of the mutants on TNF release from Murine macrophage-like RAW 264.7 cells (ATCC) was carried out as follows. RAW 264.7 cells were cultured in RPMI 1640 medium (Life Technologies, Grand Island N.Y.) supplemented with 10% fetal bovine serum (Gemini, Catabasas, Calif.), penicillin and streptomycin (Life Technologies). Polymyxin (Sigma, St. Louis, Mo.) was added at 100 units/ml to suppress the activity of any contaminating LPS. Cells were incubated with 1 μg/ml of full length (wild-type) HMG1 and each HMG1 mutant protein in Opti-MEM I medium for 8 hours, and conditioned supernatants (containing TNF which had been released from the cells) were collected and TNF released from the cells was measured by a standard murine fibroblast L929 (ATCC) cytotoxicity bioassay (Bianchi et al., supra) with the minimum detectable concentration of 30 pg/ml. Recombinant mouse TNF was obtained from R & D Systems Inc., (Minneapolis, Minn.) and used as control in these experiments. The results of this study are shown in FIG. 1. Data in FIG. 1 are all presented as mean+SEM unless otherwise indicated. (N=6-10).

As shown in FIG. 1, wild-type HMG1 and carboxyl-truncated HMG1 significantly stimulated TNF release by monocyte cultures (murine macrophage-like RAW 264.7 cells). The B box was a potent activator of monocyte TNF release. This stimulating effect of the B box was specific, because A box only weakly activated TNF release.

### EXAMPLE 3

### HMG1 B Box Protein Promotes Cytokine Activity in a Dose Dependent Manner

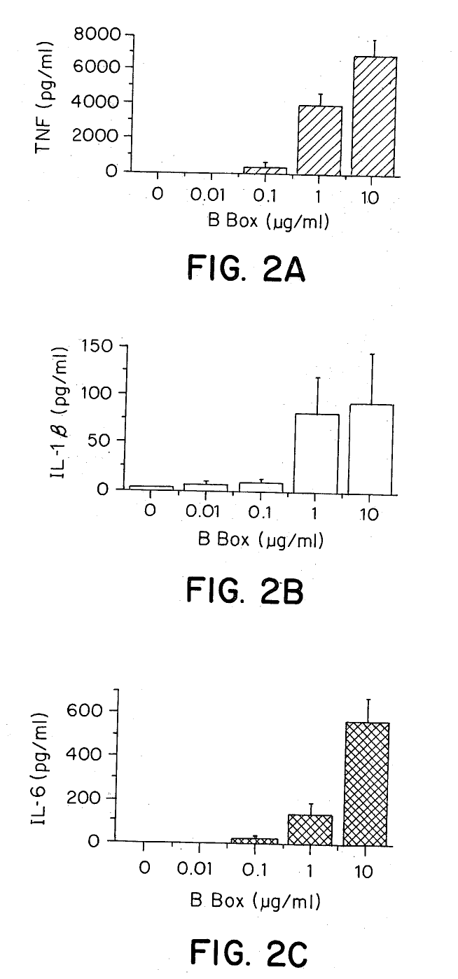

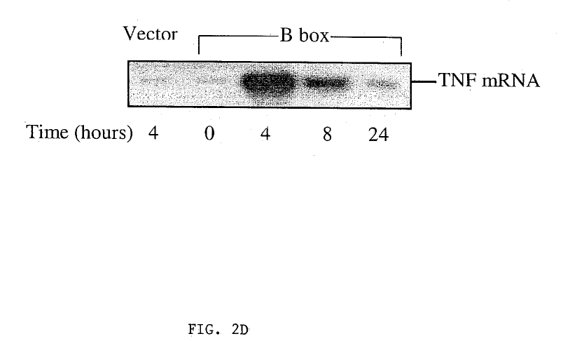

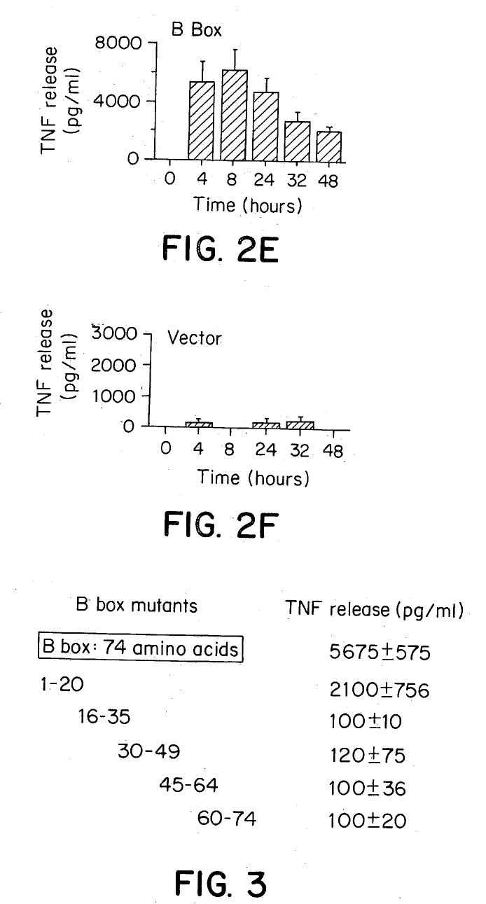

To further examine the effect of HMG1 B box on cytokine production, varying amounts of HMG1 B box were evaluated for the effects on TNF, IL-1B, and IL-6 production in murine macrophage-like RAW 264.7 cells. RAW 264.7 cells were stimulated with B box protein at 0-10 μg/ml, as indicated in FIGS. 2A-2C for 8 hours. Conditioned media were harvested and measured for TNF, IL-1β and IL-6 levels. TNF levels were measured as described herein, and IL-1β and IL-6 levels were measured using the mouse IL-1β and IL-6 enzyme-linked immunosorbent assay (ELISA) kits (R&D System Inc., Minneapolis, Minn.) and N>5 for all experiments. The results of the studies are shown in FIGS. 2A-2C.

As shown in FIG. 2A, TNF release from RAW 264.7 cells increased with increased amounts of B box administered to the cells. As shown in FIG. 2B, addition of 1 μg/ml or 10 μg/ml of B box resulted in increased release of IL-1β from RAW 264.7 cells. In addition, as shown in FIG. 2C, IL-6 release from RAW 264.7 cells increased with increased amounts of B box administered to the cells.

The kinetics of B box-induced TNF release was also examined. TNF release and TNF mRNA expression was measured in RAW 264.7 cells induced by B box polypeptide or GST tag polypeptide only used as a control (vector) (10 μg/ml) for 0 to 48 hours. Supernatants were analyzed for TNF protein levels by an L929 cytotoxicity assay (N=3-5) as described herein. For mRNA measurement, cells were plated in 100 mm plate and treated in Opti-MEM I medium containing B box polypeptide or the vector alone for 0, 4, 8, or 24 hours, as indicated in FIG. 2D. The vector only sample was assayed at the 4 hour time point. Cells were scraped off the plate and total RNA was isolated by RNAzol B method in accordance with the manufacturer's instructions (Tel-Test “B”, Inc., Friendswood, Tex.). TNF (287 bp) was measured by RNase protection assay (Ambion, Austin, Tex.). Equal loading and the integrity of RNA was verified by ethidium bromide staining of the RNA sample on agarose-formaldehyde gel. The results of the RNase protection assay are shown in FIG. 2D. As shown in FIG. 2D, B box activation of monocytes occurred at the level of gene transcription, because TNF mRNA was increased significantly in monocytes exposed to B box protein (FIG. 2B). TNF mRNA expression was maximal at 4 hours and decreased at 8 and 24 hours. The vector only control (GST tag) showed no effect on TNF mRNA expression. A similar study was carried out measuring TNF protein released from RAW 264.7 cells 0, 4, 8, 24, 32 or 48 hours after administration of B box or vector only (GST tag), using the L929 cytotoxicity assay described herein. Compared to the control (medium only), B box treatment stimulated TNF protein expression (FIG. 2F) and vector alone (FIG. 2E) did not. Data are representative of three separate experiments. Together these data indicate that the HMG1 B box domain has cytokine activity and is responsible for the cytokine stimulating activity of full length HMG1.

In summary, the HMG1 B box dose-dependently stimulated release of TNF, IL-1β and IL-6 from monocyte cultures (FIGS. 2A-2C), in agreement with the inflammatory activity of full length HMG1 (Andersson et al., J. Exp. Med. 192:565-570, 2000). In addition, these studies indicate that maximum TNF protein release occurred within 8 hours (FIG. 2F). This delayed pattern of TNF release is similar to TNF release induced by HMG1 itself, and is significantly later than the kinetics of TNF induced by LPS (Andersson et al., supra).

### EXAMPLE 4

### The First 20 Amino Acids of the HMG1 B Box Stimulate TNF Activity

The TNF-stimulating activity of the HMG1 B box was further mapped. This study was carried out as follows. Fragments of the B box were generated using synthetic peptide protection techniques, as described herein. Five HMG1 B box fragments (from SEQ ID NO:20), containing amino acids 1-20, 16-25, 30-49, 45-64, or 60-74 of the HMG1 B box were generated, as indicated in FIG. 3. RAW 264.7 cells were treated with B box (1 μg/ml) or a synthetic peptide fragment of the B box (10 μg/ml), as indicated in FIG. 3 for 10 hours and TNF release in the supernatants was measured as described herein. Data shown are mean±SEM, (n=3 experiments, each done in duplicate and validated using 3 separate lots of synthetic peptides). As shown in FIG. 3, TNF-stimulating activity was retained by a synthetic peptide corresponding to amino acids 1-20 of the HMG1 B box of SEQ ID NO:20 (fkdpnapkrlpsafflfcse; SEQ ID NO:16). The TNF stimulating activity of the 1-20-mer was less potent than either the full length synthetic B box (1-74-mer), or full length HMG1, but the stimulatory effects were specific because the synthetic 20-mers for amino acid fragments containing 16-25, 30-49, 45-64, or 60-74 of the HMG1 B box did not induce TNF release. These results are direct evidence that the macrophage stimulating activity of the B box specifically maps to the first 20 amino acids of the HMG B box domain of SEQ ID NO:20). This B box fragment can be used in the same manner as a polypeptide encoding a full length B box polypeptide, for example, to stimulate releases of a proinflammatory cytokine, or to treat a condition in a patient characterized by activation of an inflammatory cytokine cascade.

### EXAMPLE 5

### HMG1 A Box Protein Antagonizes HMG1 Induced Cytokine Activity in a Dose Dependent Manner

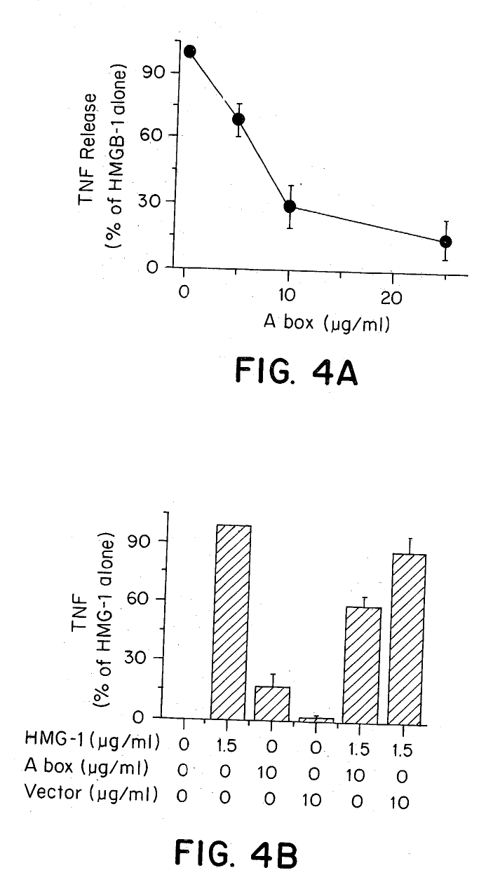

Weak agonists are by definition antagonists. Since the HMG1 A box only weakly induced TNF production, as shown in FIG. 1, the ability of HMG1 A box to act as an antagonist of HMG1 activity was evaluated. This study was carried out as follows. Sub-confluent RAW 264.7 cells in 24-well dishes were treated with HMG1 (1 μg/ml) and 0, 5, 10, or 25 μg/ml of A box for 16 hours in Opti-MEM I medium in the presence of polymyxin B (100 units/ml). The TNF-stimulating activity (assayed using the L929 cytotoxicity assay described herein) in the sample receiving no A box was expressed as 100%, and the inhibition by A box was expressed as percent of HMG1 alone. The results of the effect of A box on TNF release from RAW 264.7 cells is shown in FIG. 4A. As shown in FIG. 4A, the A box dose-dependently inhibited HMG1 induced TNF release with an apparent EC₅₀ of approximately 7.5 μg/ml. Data in FIG. 4A are presented as mean±SD (n=2-3 independent experiments).

### EXAMPLE 6

### HMG1 A Box Protein Inhibits Full Length HMG1 and HMG1 B Box Cytokine Activity

Antagonism of full length HMG1 activity by HMG1 A box or GST tag (vector control) was also determined by measuring TNF release from RAW 264.7 macrophage cultures stimulated by co-addition of A box with full length HMG1. RAW 264.7 macrophage cells (ATCC) were seeded into 24-well tissue culture plates and used at 90% confluence. The cells were treated with HMG1, and/or A boxes as indicated for 16 hours in Optimum I medium (Life Technologies, Grand Island, N.Y.) in the presence of polymyxin B (100 units/ml, Sigma, St. Louis, Mo.) and supernatants were collected for TNF measurement (mouse ELISA kit from R&D System Inc, Minneapolis, Minn.). TNF-inducing activity was expressed as a percentage of the activity achieved with HMG-1 alone. The results of these studies are shown in FIG. 4B. FIG. 4B is a histogram of the effect of HMG1, alone, A box alone, Vector (control) alone, HMG1 in combination with A box, and HMG1 in combination with vector. As shown in FIG. 4B, HMG1 A box significantly attenuated the TNF stimulating activity of full length HMG1.

### EXAMPLE 7

### HMG1 A Box Protein Inhibits HMG1 Cytokine Activity by Binding to It

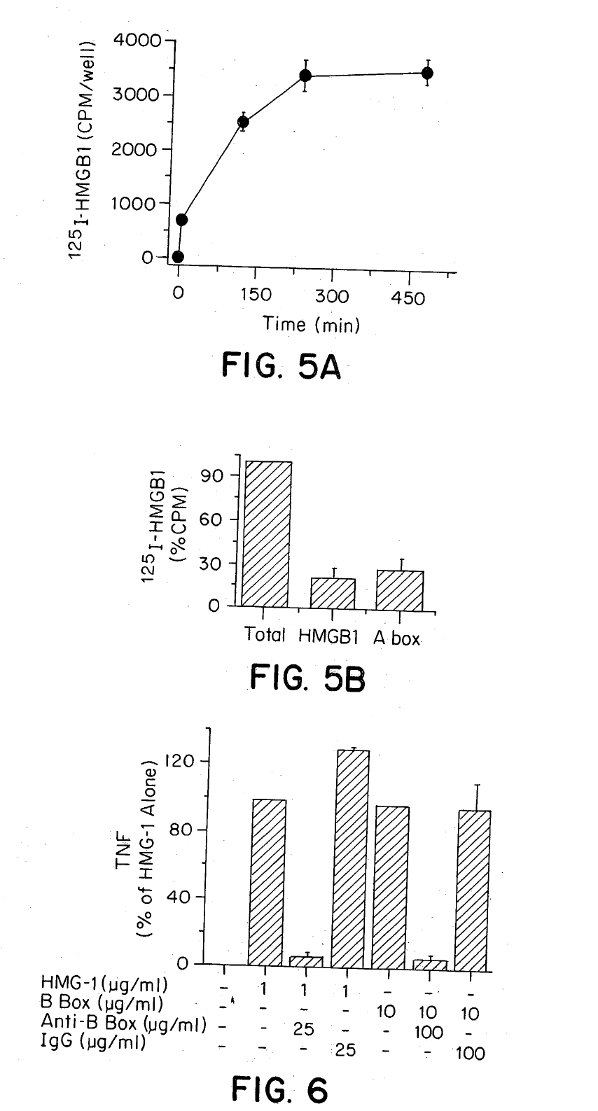

To determine whether the HMG1 A box acts as an antagonist by displacing HMG1 binding, ¹²⁵I-labeled -HMG1 was added to macrophage cultures and binding was measured at 4° C. after 2 hours. Binding assays in RAW 264.7 cells were performed as described herein. ¹²⁵I-HMG1 binding was measured in RAW 264.7 cells plated in 24-well dishes for the times indicated in FIG. 5A. Specific binding shown equals total cell-associated ¹²⁵I-HMG1 (CPM/well) minus cell associated CPM/well in the presence of 5,000 fold molar excess of unlabeled HMG1. FIG. 5A is a graph of the binding of ¹²⁵I-HMG1 over time. As shown in FIG. 5A, HMG1 exhibited saturable first order binding kinetics. The specificity of binding was assessed as described in Example 1.

In addition, ¹²⁵I-HMG-1 binding was measured in RAW 264.7 cells plated on 24-well dishes and incubated with ¹²⁵I HMG1 alone or in the presence of unlabeled HMG1 or A box. The results of this binding assay are shown in FIG. 5B. Data represents mean±SEM from 3 separate experiments. FIG. 5B is a histogram of the cell surface binding of ¹²⁵I-HMGB1 in the absence of unlabeled HMGB1 or HMGB1 (HMG1) A box, or in the presence of 5,000 molar excess of unlabeled HMGB1 or HMGB1 A box, measured as a percent of the total CPM/well. In FIG. 5B, “Total” equals counts per minutes (CPM)/well of cell associated ¹²⁵I-HMGB1 in the absence of unlabeled HMGB1 or A box for 2 hours at 4° C. “HMGB1” or “A box” equals to CPM/well of cell-associated ¹²⁵I-HMGB1 in the presence of 5,000 molar excess of unlabeled HMGB1 or A box. The data are expressed as the percent of total counts obtained in the absence of unlabeled HMGB1 proteins (2,382,179 CPM/well). These results indicate that the HMG1 A box is a competitive antagonist of HMG1 activity in vitro that inhibits the TNF-stimulating activity of HMG1.

### EXAMPLE 8

### Inhibition of Full Length HMG1 and HMG1 B Box Cytokine Activity by Anti-B Box Polyclonal Antibodies.

The ability of antibodies directed against the HMG1 B box to modulated the effect of full length or HMG1 B box was also assessed. Affinity purified antibodies directed against the HMG1 B box (B box antibodies) were generated as described herein and using standard techniques. To assay the effect of the antibodies on HMG1- or HMG1 B box- induced TNF release from RAW 264.7 cells, sub-confluent RAW 264.7 cells in 24-well dishes were treated with HMG-1 (1 μg/ml) or HMG1 B box (10 μg/ml) for 10 hours with or without anti-B box antibody (25 μg/ml or 100 μg/ml antigen affinity purified, Cocalico Biologicals, Inc., Reamstown, Pa.) or non-immune IgG (25 μg/ml or 100 μg/ml; Sigma) added. TNF release from the RAW 264.7 cells was measured using the L929 cytotoxicity assay as described herein. The results of this study are shown in FIG. 6, which is a histogram of TNF released by RAW 264.7 cells administered nothing, 1 μg/ml HMG1, 1 μg/ml HMG1 plus 25 μg/ml anti-B box antibody, 1 μg/ml HMG1 plus 25 μg/ml IgG (control), 10 μg/ml B-box, 10 μg/ml B-box plus 100 μg/ml anti-B box antibody or 10 μg/ml B-box plus 100 μg/ml IgG (control). The amount of TNF released from the cells induced by HMG1 alone (without addition of B box antibodies) was set as 100%, the data shown in FIG. 6 are the results of 3 independent experiments. As shown in FIG. 6, affinity purified antibodies directed against the HMG1 B box significantly inhibited TNF release induced by either full length HMG1 or the HMG1 B box. These results indicate that such an antibody can be used to modulate HMG1 function.

### EXAMPLE 9

### HMG1 B Box Protein Is Toxic to D-galactosamine-Sensitized Balb/c Mice

To investigate whether the HMG1 B box has cytokine activity in vivo, we administered HMG1 B box protein to unanesthetized Balb/c mice sensitized with D-galactosamine (D-gal), a model that is widely used to study cytokine toxicity (Galanos et al., supra). Briefly, mice (20-25 gram, male, Harlan Sprague-Dawley, Indianapolis, Ind.) were intraperitoneally injected with D-gal (20 mg) (Sigma) and B box (0.1 mg/ml/mouse or 1 mg/ml/mouse) or GST tag (vector; 0.1 mg/ml/mouse or 1 mg/ml/mouse), as indicated in Table 1. Survival of the mice was monitored up to 7 days to ensure no late death occurred. The results of this study are shown in Table 1.

TABLE 1
Toxicity of HMG1 B box on D-galactosamine-sensitized Balb/c Mice

| | Treatment | Alive/total |
|---|---|---|
| Control | — | 10/10 |
| Vector | 0.1 mg/mouse | 2/2 |
| | 1 mg/mouse | 3/3 |
| B box | 0.1 mg/mouse | 6/6 |
| | 1 mg/mouse | 2/8* |

P < 0.01 versus vector alone as tested by Fisher's Exact Test

The results of this study showed that the HMG1 B box was lethal to D-galactosamine-sensitized mice in a dose-dependent manner. In all instances in which death occurred, it occurred within 12 hours. Lethality was not observed in mice treated with comparable preparations of the purified GST vector protein devoid of B box.

### EXAMPLE 10

### Histology of D-galactosamine-Sensitized Balb/c Mice or C3H/HeJ Mice Administered HMG1 B Box Protein

To further assess the lethality of the HMG1 B box protein in vivo the HMG1 B box was again administered to D-galactosamine-sensitized Balb/c mice. Mice (3 per group) received D-gal (20 mg/mouse) plus B box or vector (1 mg/mouse) intraperitoneally for 7 hours and were then sacrificed by decapitation. Blood was collected, and organs (liver, heart, kidney and lung) were harvested and fixed in 10% formaldehyde. Tissue sections were prepared with hematoxylin and eosin staining for histological evaluation (Criterion Inc., Vancouver, Canada). The results of these studies are shown in FIGS. 7A-7J, which are scanned images of hematoxylin and eosin stained kidney sections (FIG. 7A), myocardium sections (FIG. 7C), lung sections (FIG. 7E), and liver sections (FIGS. 7G and 7I) obtained from an untreated mouse and kidney sections (FIG. 7B), myocardium sections (FIG. 7D), lung sections (FIG. 7F), and liver sections (FIGS. 7H and 7J) obtained from mice treated with the HMG1 B box. Compared to the control mice, B box treatment caused no abnormality in kidneys (FIGS. 7A and 7B) and lungs (FIGS. 7E and 7F). The mice had some ischemic changes and loss of cross striation in myocardial fibers in the heart (FIGS. 7C and 7D as indicated by the arrow in FIG. 7D). Liver showed most of the damage by the B box as illustrated by active hepatitis (FIGS. 7G-7J). In FIG. 7J, hepatocyte dropouts are seen surrounded by accumulated polymorphonuclear leukocytes. The arrows in FIG. 7J point to the sites of polymorphonuclear accumulation (dotted) or apoptotic hepatocytes (solid). Administration of HMG1 B box in vivo also stimulated significantly increased serum levels of IL-6 (315+93 vs.20+7 pg/ml, B box vs. control, p<0.05) and IL-1β (15+3 vs. 4+1 pg/ml, B box vs. control, p<0.05).

Administration of B box protein to C3H/HeJ mice (which do not respond to endotoxin) was also lethal, indicating that HMG1 B box is lethal in the absence of LPS signal transduction. Hematoxylin and eosin stained sections of lung and kidney collected 8 hours after administration of B box revealed no abnormal morphologic changes. Examination of sections from the heart however, revealed evidence of ischemia with loss of cross striation associated with amorphous pink cytoplasm in myocardial fibers. Sections from liver showed mild acute inflammatory responses, with some hepatocyte dropout and apoptosis, and occasional polymorphonuclear leukocytes. These specific pathological changes were comparable to those observed after administration of full length HMG1 and confirm that the B box alone can recapitulate the lethal pathological response to HMG1 in vivo.

To address whether the TNF-stimulating activity of HMG1 contributes to the mediation of lethality by B box, we measured lethality in TNF knock-out mice (TNF-KO, Nowak et al., Am. J. Physiol. Regul. Integr. Comp. Physiol. 278: R1202-R1209, 2000) and the wild-type controls (B6x129 strain) sensitized with D-galactosamine (20 mg/mouse) and exposed to B box (1 mg/mouse, injected intraperitoneally). The B box was highly lethal to the wild-type mice (6 dead out of nine exposed) but lethality was not observed in the TNF-KO mice treated with B box (0 dead out of 9 exposed, p<0.05 v. wild type). Together with the data from the RAW 264.7 macrophage cultures, described herein, these data now indicate that the B box of HMG1 confers specific TNF-stimulating cytokine activity.

### EXAMPLE 11

### HMG1 Protein Level is Increased in Septic Mice

To examine the role of HMG1 in sepsis, we established sepsis in mice and measured serum HMG1 using a quantitative immunoassay described previously (Wang et al., supra). Mice were subjected to cecal ligation and puncture (CLP), a well characterized model of sepsis caused by perforating a surgically-created cecal diverticulum, that leads to polymicrobial peritonitis and sepsis (Fink and Heard, supra; Wichmann et al., supra; and Remick et al., supra). Serum levels of HMG1 were then measured (Wang et al., supra). FIG. 8 shows the results of this study in a graph that illustrates the levels of HMG1 in mice 0 hours, 8 hours, 18 hours, 24 hours, 48 hours, and 72 hours after subjection to CLP. As shown in FIG. 8, serum HMG1 levels were not significantly increased for the first eight hours after cecal perforation, then increased significantly after 18 hours (FIG. 8). Increased serum HMG1 remained at elevated plateau levels for at least 72 hours after CLP, a kinetic profile that is quite similar to the previously described, delayed HMG1 kinetics in endotoxemia (Wang et al., supra). This temporal pattern of HMG1 release corresponded closely to the development of signs of sepsis in the mice. During the first eight hours after cecal perforation the animals were observed to be mildly ill, with some diminished activity and loss of exploratory behavior. Over the ensuing 18 hours the animals became gravely ill, huddled together in groups with piloerection, did not seek water or food, and became minimally responsive to external stimuli or being examined by the handler.

### EXAMPLE 12

### Treatment of Septic Mice with HMG1 A Box Protein Increases Survival of Mice

To determine whether the HMG1 A box can inhibit the lethality of HMG1 during sepsis, mice were subjected to cecal perforation and treated by administration of A box beginning 24 hours after the onset of sepsis. CLP was performed on male Balb/c mice as described herein. Animals were randomly grouped, with 15-25 mice per group. The HMG1 A box (60 or 600 μg/mouse each time) or vector (GST tag, 600 μg/mouse) alone was administered intraperitoneally twice daily for 3 days beginning 24 hours after CLP. Survival was monitored twice daily for up to 2 weeks to ensure no late death occurred. The results of this study are illustrated in FIG. 9, which is a graph of the effect of vector (GST; control) 60 μg/mouse or 600 μg/mouse on survival over time (*P<0.03 vs. control as tested by Fisher's exact test). As shown in FIG. 9, administration of the HMG1 A box significantly rescued mice from the lethal effects of sepsis, and improved survival from 28% in the animals treated with protein purified from the vector protein (GST) devoid of the A box, to 68% in animals receiving A box (P<0.03 by Fischer's exact test). The rescuing effects of the HMG1 A box in this sepsis model were A box dose-dependent; animals treated with 600 μg/mouse of A box were observed to be significantly more alert, active, and to resume feeding behavior as compared to either controls treated with vector-derived preparations, or to animals treated with only 60 μg A box. The latter animals remained gravely ill, with depressed activity and feeding for several days, and most died.

### EXAMPLE 13

### Treatment of Septic Mice with Anti-HMG1 Antibody Increases Survival of Mice

Passive immunization of critically ill septic mice with anti-HMG1 antibodies was also assessed. In this study, male Balb/c mice (20-25 gm) were subjected to CLP, as described herein. Affinity purified anti-HMG1 B box polyclonal antibody or rabbit IgG (as control) was administered at 600 μg/mouse beginning 24 hours after the surgery, and twice daily for 3 days. Survival was monitored for 2 weeks. The results of this study are shown in FIG. 10A which is a graph of the survival of septic mice treated with either a control antibody or an anti-HMG1 antibody. The results show that anti-HMG1 antibodies administered to the mice 24 hours after the onset of cecal perforation significantly rescued animals from death as compared to administration of non-immune antibodies (p<0.02 by Fisher's exact test). Within 12 hours after administration of anti-HMG1 antibodies, treated animals showed increased activity and responsiveness as compared to controls receiving non-immune antibodies. Whereas animals treated with non-immune antibodies remained huddled, ill kempt, and inactive, the treated animals improved significantly and within 48 hours resumed normal feeding behavior. Anti-HMG1 antibodies did not suppress bacterial proliferation in this model, because we observed comparable bacterial counts (CFU, the aerobic colony forming units) from spleen 31 hours after CLP in the treated animals as compared to animals receiving irrelevant antibodies (control bacteria counts=3.5±0.9×10⁴ CFU/g; n=7). Animals were monitored for up to 2 weeks afterwards, and late deaths were not observed, indicating that treatment with anti-HMG1 conferred complete rescue from lethal sepsis, and did not merely delay death.

To our knowledge, no other specific cytokine-directed therapeutic is as effective when administered so late after the onset of sepsis. By comparison, administration of anti-TNF actually increases mortality in this model, and anti-MIF antibodies are ineffective if administered more than 8 hours after cecal perforation (Remick et al, supra; and Calandra et al., Nature Med. 6:164-170, 2000). These data demonstrate that HMG1 can be targeted as late as 24 hours after cecal perforation in order to rescue lethal cases of established sepsis.

In another example of the rescue of endotoxemic mice using anti-B box antibodies, anti-HMG1 B box antibodies were evaluated for their ability to rescue LPS-induced septic mice. Male Balb/c mice (20-25 gm, 26 per group) were treated with an LD75 dose of LPS (15 mg/kg) injected intraperitoneally (IP). Anti-HMG1 B box or non-immune rabbit serum (0.3 ml per mouse each time, IP) was given at time 0, +12 hours and +24 hours after LPS administration. Survival of mice was evaluated over time. The results of this study are shown in FIG. 10B, which is a graph of the survival of septic mice administered anti-HMG1 B box antibodies or non-immune serum. As shown in FIG. 10B, anti-HMG1 B box antibodies improved survival of the septic mice.

### EXAMPLE 14

### Inhibition of HMG1 Signaling Pathway Using an Anti-RAGE Antibody

Previous data implicated RAGE as an HMG1 receptor that can mediate neurite outgrowth during brain development and migration of smooth muscle cells in wound healing (Hori et al. J. Biol. chem. 270:25752-25761, 1995; Merenmies et al. J. Biol. Chem. 266:16722-16729, 1991; and Degryse et al., J. Cell Biol. 152:1197-1206, 2001). We measured TNF release in RAW 264.7 cultures stimulated with HMG1 (1 μg/ml), LPS (0.1 μg/ml), or HMG1 B box (1 μg/ml) in the presence of anti-RAGE antibody (25 μg/ml) or non-immune IgG (25 μg/ml). Briefly, the cells were seeded into 24- well tissue culture plates and used at 90% confluence. LPS (E. coli 0111:B4, Sigma, St. Louis, Mo.) was sonicated for 20 minutes before use. Cells were treated with HMG1 (1 μg/ml), LPS (0.1 μg/ml), or HMG1 B box (1 μg/ml) in the presence of anti-RAGE antibody (25 μg/ml) or non-immune IgG (25 μg/ml) as indicated in FIG. 11A for 16 hours in serum-free Opti-MEM I medium (Life Technologies) and supernatants were collected for TNF measurement using the L929 cytotoxicity assay described herein. IgG purified polyclonal anti-RAGE antibody (Catalog No.sc-8230, N-16, Santa Cruz Biotech, Inc., Santa Cruz, Calif.) was dialyzed extensively against PBS before use. The results of this study are shown in FIG. 11A, which is a histogram of the effects of HMG1, LPS, or HMG1 B box in the presence of anti-RAGE antibodies or non-immune IgG (control) on TNF release from RAW 264.7 cells. As shown in FIG. 11A, compared to non-immune IgG, anti-RAGE antibody significantly inhibited HMG1 B box-induced TNF release. This suppression was specific, because anti-RAGE did not significantly inhibit LPS-stimulated TNF release. Notably, the maximum inhibitory effect of anti-RAGE decreased HMG-1 signaling by only 40%, suggesting that other signal transduction pathways may participate in HMG1 signaling.

To examine the effects of HMG1 or HMG1 B Box on the NF-kB-dependent ELAM promoter, the following experiment was carried out. RAW 264.7 macrophages were transiently co-transfected with an expression plasmid encoding a murine MyD 88-dominant-negative (DN) mutant (corresponding to amino acids 146-296), or empty vector, plus a luciferase reporter plasmid under the control of the NF-kB-dependent ELAM promoter, as described by Means et al. (J. Immunol. 166:4074-4082, 2001). A portion of the cells were then stimulated with full-length HMG1 (100 ng/ml), or purified HMG1 B box (10 μg/ml), for 5 hours. Cells were then harvested and luciferase activity was measured, using standard methods. All transfections were performed in triplicate, repeated at least three times, and a single representative experiment is shown in FIG. 11B. As shown in FIG. 11B, HMG1 stimulated luciferase activity in samples that were not co-transfected with the MyD 88 dominant negative, and the level of stimulation was decreased in samples that were co-transfected with the MyD 88 dominant negative. This effect was also observed in samples administered HMG B box.

The effect of HMG1 or HMG1 B box on NF-kB activation was also examined. CHO reporter cell lines that constitutively express human Toll-like receptor 2 (TLR2) or Toll-like receptor 4 (TLR4) have been previously described (Means et al., J. Immunology, 163:3920-3927, 1999). These reporter lines also contain a stably transfected ELAM-CD25 reporter gene, and express human CD25 on their surface as a consequence of NF-kB activation. CHO/TLR2 and CHO/TLR4 cells were stimulated with IL-1 (10 ng/ml), purified full-length HMG-1 (100 ng/ml), or purified B box (10 μg/ml) for 18 hours. Following stimulation, cells were stained with a PE-labeled anti-CD25 monoclonal antibody and surface expression of CD25 was measured by flow cytometry. The results of this study are shown ib FIG. 11C. Data are expressed as the ratio (fold-activation) of the percent of CD25⁺ cells in unstimulated and stimulated cell populations that were gated to exclude the lowest 5% of cells based on mean FL1 fluorescence. In CHO/TLR4 cells, stimulation with each of HMG1 and HMG1 B box resulted in decreased CD25 expression compared to the CHO/TLR2 samples.

The effect of anti-RAGE antibodies, anti-TLR2 antibodies, a combination of anti-RAGE antibodies and anti-TLR2 antibodies or IgG, on HMG-1-mediated TNF release in RAW 264.7 cells was also determined. RAW 264.7 cells were seeded into 24-well tissue culture plates and used at 90% confluence. Cells were incubated with HMG-1 with or without anti-RAGE antibody (Cat# sc-8230, Santa Cruz Biotech Inc., Santa Cruz, Calif.), anti-TLR2 antibody (Affinity-purified polyclonal antibody, Cat # sc-12504, D17, Santa Cruz) or IgG (non-immune IgG, Sigma, St. Louis, Mo.) in Optimum I medium (Life Technologies, Grand Island, N.Y.) in the presence of polymyxin B (100 units/ml, Sigma, St. Louis, Mo.) for 16 hours. Antibodies were dialyzed against PBS to remove sodium azide before use. Conditioned media were collected and a TNF ELISA was performed, using standard ELISA methods. Data (n=3) were expressed as a percentage of the activity achieved with HMG-1 alone. The results of this study are shown in FIG. 11D. Both anti-RAGE and anti-TLR2 antibodies significantly (*P<0.05) inhibited HMG-1-mediated TNF release. Combination of the 2 antibodies had additive effects in inhibiting TNF release whereas IgG was irrelevant.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of inhibiting release of a proinflammatory cytokine from a mammalian cell, comprising administering to said cell an antibody that binds a Toll-like 2 receptor.

2. The method of claim 1, wherein said cell is a macrophage.

3. The method of claim 1, wherein said cytokine is TNF.

4. The method of claim 1, wherein said release is HMG-1 mediated.

5. A method of inhibiting a TNF-mediated condition in a patient, comprising administering to said patient an antibody that binds a Toll-like 2 receptor.

6. The method of claim 5, wherein said condition is selected from the group consisting of appendicitis, peptic, gastric and duodenal ulcers, peritonitis, pancreatitis, ulcerative, pseudomembranous, acute and ischemic colitis, diverticulitis, epiglottitis, achalasia, cholangitis, cholecystitis, hepatitis, Crohn's disease, enteritis, Whipple's disease, asthma, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopicsilicovolcanoconiosis, alvealitis, bronchiolitis, pharyngitis, pleurisy, sinusitis, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcets's syndrome, allograft rejection, graft-versus-host disease, Type I diabetes, ankylosing spondylitis, Berger's disease, Retier's syndrome, and Hodgkins disease.

7. The method of claim 5, wherein said condition is selected from the group consisting of peritonitis, pancreatitis, ulcerative colitis, Crohn's disease, asthma, organ ischemia, reperfusion injury, sepsis, cachexia, burns, myocardial ischemia, adult respiratory distress syndrome, multiple sclerosis, rheumatoid arthritis, systemic lupus erythematous, Behcet's syndrome, allograft rejection and graft-versus-host disease.

8. The method of claim 7, wherein said condition is sepsis.

9. The method of claim 7, wherein said condition is rheumatoid arthritis.

10. The method of claim 7, wherein said condition is systemic lupus erythematous.

11. The method of claim 7, wherein said condition is allograft rejection.

12. The method of claim 7, wherein said condition is peritonitis.

13. The method of claim 7, wherein said condition is adult respiratory distress syndrome.

14. The method of claim 7, wherein said condition is multiple sclerosis.

15. The method of claim 7, wherein said condition is asthma.

16. The method of claim 7, wherein said condition is pancreatitis.

17. The method of claim 7, wherein said condition is organ ischemia.

18. The method of claim 7, wherein said condition is reperfusion injury.

19. The method of claim 7, wherein said condition is ulcerative colitis.

20. The method of claim 7, wherein said condition is Crohn's disease.


SEQUENCE LISTING (23 sequences)

SEQ ID NO: 1 - protein, 215 aa, Homo Sapien
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 2 - protein, 215 aa, Mus Musculus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEEEEDEDEEEDDDDE

SEQ ID NO: 3 - protein, 209 aa, Homo Sapien
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 4 - protein, 54 aa, Homo Sapien
PDASVNFSEFSKKCSERWKTMSAKEKGKFEDMAKADKARYEREMKTYIPPKGET

SEQ ID NO: 5 - protein, 69 aa, Homo Sapien
NAPKRPPSAFFLFCSEYRPKIKGEHPGLSIGDVAKKLGEMWNNTAADDKQPYEKKAAKLKEKYEKDIAA

SEQ ID NO: 6 - DNA, 22 bp, Homo Sapien
gatgggcaaaggagatcctaag

SEQ ID NO: 7 - DNA, 29 bp, Homo Sapien
gcggccgcttattcatcatcatcatcttc

SEQ ID NO: 8 - DNA, 22 bp, Homo Sapien
gatgggcaaaggagatcctaag

SEQ ID NO: 9 - DNA, 32 bp, Homo Sapien
gcggccgctcacttgcttttttcagccttgac

SEQ ID NO: 10 - DNA, 21 bp, Homo Sapien
gagcataagaagaagcaccca

SEQ ID NO: 11 - DNA, 32 bp, Homo Sapien; identical to SEQ ID NO: 9

SEQ ID NO: 12 - DNA, 24 bp, Homo Sapien
aagttcaaggatcccaatgcaaag

SEQ ID NO: 13 - DNA, 32 bp, Homo Sapien
gcggccgctcaatatgcagctatatccttttc

SEQ ID NO: 14 - DNA, 22 bp, Homo Sapien
gatgggcaaaggagatcctaag

SEQ ID NO: 15 - DNA, 24 bp, Homo Sapien
tcacttttttgtctcccctttggg

SEQ ID NO: 16 - protein, 20 aa, Homo Sapien
FKDPNAPKRLPSAFFLFCSE

SEQ ID NO: 17 - protein, 54 aa, Homo Sapien
PDSSVNFAEFSKKCSERWKTMSAKEKSKFEDMAKSDKARYDREMKNYVPPKGDK

SEQ ID NO: 18 - protein, 216 aa, Homo Sapien [sequence omitted]

SEQ ID NO: 19 - protein, 182 aa, Homo Sapien
MGKGDPKKPTGKMSSYAFFVQTCREEHKKKHPDASVNFSEFSKKCSERWKTMSAKEKGKFEDMAKADKARYEREMKTYIPPKGETKKKFKDPNAPKRLPSAFFLFCSEYRPKIKGEHPGLSIGDVAKKLGEMWNNTAADDKQPYEKKAAKLKEKYEKDIAAYRAKGKPDAAKKGVVKAEKSK

SEQ ID NO: 20 - protein, 74 aa, Homo Sapien
FKDPNAPKRLPSAFFLFCSEYRPKIKGEHPGLSIGDVAKKLGEMWNNTAADDKQPYEKKAAKLKEKYEKDIAAY

SEQ ID NO: 21 - protein, 85 aa, Homo Sapien
MGKGDPKKPTGKMSSYAFFVQTCREEHKKKHPDASVNFSEFSKKCSERWKTMSAKEKGKFEDMAKADKARYEREMKTYIPPKGET

SEQ ID NO: 22 - protein, 77 aa, Homo Sapien
PTGKMSSYAFFVQTCREEHKKKHPDASVNFSEFSKKCSERWKTMSAKEKGKFEDMAKADKARYEREMKTYIPPKGET

SEQ ID NO: 23 - protein, 20 aa, Homo Sapien
NAPKRPPSAFFLFCSEYRPK